(19) United States
(12) Patent Application Publication
(10) Pub. No.: US 2025/0332162 A1
(43) Pub. Date: Oct. 30, 2025

(54) INHIBITING PURINE BIOSYNTHESIS TO INCREASE FAVIPIRAVIR POTENCY AGAINST RNA VIRUS INFECTIONS

(71) Applicant: VIRIOM, INC., San Diego, CA (US)

(72) Inventors: Haishan Li, North Potomac, MD (US); Juan Carlos Zapata, Ijamsville, MD (US); Sandra Margarita Medina Moreno, Rockville, MD (US); Charles David Pauza, Gaithersburg, MD (US)

(21) Appl. No.: 18/866,525

(22) PCT Filed: May 13, 2023
(86) PCT No.: PCT/US2023/066978

§ 371 Date: Nov. 15, 2024

Related U.S. Application Data
(60) Provisional application No. 63/342,335, filed on May 16, 2022.

(51) Int. Cl. A61K 31/4965; A61K 31/5377; A61K 31/7056; A61P 31/16

(52) U.S. Cl. CPC A61K 31/4965; A61K 31/5377; A61K 31/7056; A61P 31/16

(57) ABSTRACT

The present invention is generally directed to a potent therapy for SARS-CoV-2 (CoV2) disease which may involve combinations of agents. Here we describe combinations of 2, 3 or more drugs wherein the combination inhibits CoV2 replication through one or more mechanisms of action and increases potency of nucleoside and nucleotide analog drugs through inhibition of cellular enzymes involved in purine nucleotide biosynthesis. The combinations may be delivered as individual doses, concurrent dosing, or co-formulation of 2 or more agents. The inventive aspect includes identified components, the ratios among identified components and treatment regimens for reducing morbidity and mortality of CoV2 infection. Further claimed are drug formulations and methods of delivery.

### CROSS-REFERENCE TO RELATED APPLICATIONS

This application claims priority to and benefit of U.S. Provisional Patent Application Ser. No. 63/342,335 filed May 16, 2022, entitled “INHIBITING PURINE BIOSYNTHESIS TO INCREASE FAVIPIRAVIR POTENCY AGAINST RNA VIRUS INFECTIONS”, the disclosure of which is incorporated by reference in its entirety for all purposes.

### FIELD OF INVENTION

The invention relates to the field of antiviral therapy and, in particular, to drug combinations useful for treatment and prevention of SARS-CoV-2 virus infection. The invention provides novel drug combinations for treatment or prophylaxis of SARS-CoV-2 mediated diseases including COVID19, its prodrome, and the long COVID syndrome. The invention is concerned with combination product and pharmaceutical composition that improve potency of each component while also increasing safety margins.

### BACKGROUND

The severe acute respiratory syndrome coronavirus 2 (SARS-CoV-2) emerged in 2019 within the city of Wuhan, China, as an outbreak of severe respiratory disease. This highly infectious agent spread rapidly and caused a global pandemic with many deaths and hardships. Since January 2020, there have been approximately 500,000,000 confirmed cases of COVID-19 and more than 6,000,000 deaths reported to the World Health Organization (WHO). More than 78,000,000 confirmed cases of COVID-19 and nearly 1,000,000 deaths were reported in the United States (US). Multiple vaccine development and distribution programs collectively administered >10,000,000,000 vaccine doses as of March 2022 and as of Feb. 25, 2022, more than 500,000,000 vaccine doses were administered in the US alone (https://covid19.who.int/accessed on Mar. 8, 2022).

Despite efforts to roll out vaccine programs, the COVID-19 pandemic has not been contained and there is an urgent need to supplement vaccination efforts with antiviral drug therapy. Oral antiviral drugs are particularly valuable options for COVID-19 especially if used early to slow or prevent disease progression. Several antiviral compounds have already been approved for COVID-19 on an emergency use basis in the US, European Union (EU), United Kingdom, China, Russia, and India with a number of other countries accelerating reviews for emergency use approval.

Three oral antiviral compounds for treating of COVID-19 in hospital, outpatient and community settings include Favipiravir (also known as AVIFAVIR, AVIGAN, FABIFLU), Molnupiravir (also known as Lagevrio), and the combination of Nirmatrelvir/Ritonavir (also known as Paxlovid). The three treatments were developed originally for a viral disease other than COVID-19 before being repurposed as SARS-CoV-2 antiviral agents. Because of prior experience with these drugs, it was possible to develop and provide treatments expeditiously as part of the emergency pandemic response.

Favipiravir and Molnupiravir are nucleoside analogues, share similar antiviral mechanisms of action (MoA) and may be synergistic when administered concomitantly (Abdelnabi et al, 2021). These oral prodrugs convert into non-natural phosphorylated nucleotides which are incorporated into nascent RNA chains by the RNA-dependent RNA polymerase (RdRp) of SARS-CoV-2.

Favipiravir acts predominantly as a purine (guanine) analogue, while Molnupiravir is predominantly recognized as a pyrimidine (cytidine) analogue (Soto-Acosta et al, 2021: Peng Q et al. 2021: Jin et al, 2013) and their effects can be modulated by the status of intracellular nucleoside pools (Schultz et al, 2022). Molnupiravir and Favipiravir are also chain terminators (Zhou et al, 2021). Favipiravir caused termination of Influenza A RNA chains when two consecutive Favipiravir nucleotides were incorporated into a nascent strand thus, destabilizing the active site by disrupting base stacking (Wang et al, 2021).

The nirmatrelvir drug inhibits a viral protease, and its mechanism of action is unrelated to the lethal mutagenesis drugs.

More commonly, Favipiravir incorporation escapes an editing mechanism of the SARS-CoV-2 replicase complex and is incorporated into nascent RNA as a non-natural nucleotide. Misreading of the non-natural nucleotide during subsequent RNA replication causes G to A transitions (mutations. Once the frequency of mutations passes a threshold, viral fitness is reduced, and viremia is suppressed. The mechanism of action for Molnupiravir is similar, although the active drug is more prone to causing DNA mutations than is Favipiravir. Consequently, Favipiravir is a better choice for SARS-CoV-2 therapy due mainly to having larger safety margins. The main obstacle to routine use of Favipiravir for COVID19 is the high pill burden required to achieve active drug levels. Consequently, medical professionals are seeking new drug combinations including Favipiravir to improve potency and possibly reduce the pill burden.

Several Favipiravir combinations with non-antiviral agents were clinically effective in COVID-19 patients, including those who were critically ill. A combination of Favipiravir and steroids was reported to be beneficial for preventing severe COVID-19 pneumonia when drugs were administered in the early stage of disease (Murohashi et al, 2020; Inoue et al, 2020; Shindo et al, 2021). Similar benefits in COVID-19 patients were reported with the combination of Favipiravir and Tocilizumab (Zhao H et al, 2021) and Favipiravir plus Nafamostat mesylate (Doi et al, 2020). Initial experience with combination therapy of Lopinavir/Ritonavir plus Favipiravir indicated that this combination may be another Favipiravir-based option for COVID-19 patients (Koba et al, 2020).

While these drug combinations have improved clinical outcomes of Favipiravir therapy, they are not rationally designed, do not exploit our knowledge of Favipiravir mode of action and are unlikely to demonstrate synergy, which is relevant to decreasing drug dose and improving safety margins. When non-natural nucleoside drugs are used, host pathways normally used to synthesize purine nucleotides are exploited for the steps of ribosylating the Favipiravir base and producing the active, triphosphorylated form. At each step up to and including incorporation in nascent RNA chains, the Favipiravir is competing with the pools of natural nucleotides. Thus, the pool sizes for purine nucleotides and guanine (GTP) in particular, are critical factors affecting Favipiravir potency. Reducing the cellular concentration of GTP, while administering the same doses of Favipiravir, increases chances that Favipiravir will be incorporated into nascent RNA. We can use this knowledge to improve the potency of Favipiravir once we are able to identify the step of purine biosynthesis that is amenable to inhibition and increases Favipiravir potency, select drugs capable of inhibiting the selected enzyme in purine biosynthesis, test drug combinations to detect additive or synergistic effects of 2, 3 or more drugs given together, and confirm that adequate safety margins are maintained. The instant invention provides combinations of Favipiravir and inhibitors of purine biosynthesis with increased potency and better safety margins and defines these combinations in terms of precise molar ratios most suitable for SARS-CoV-2 therapy.

### SUMMARY

A first aspect of the invention relates to a combination product of (i) inhibitor viral RNA replication and (ii) Inosine Monophosphate Nucleotide Dehydrogenase (IMPDH) inhibitor.

The present invention describes novel combinations of (i) inhibitor of viral RNA replication, and (ii) IMPDH inhibitors and their use in therapy, in particular in the treatment of infections caused by RNA viruses.

More specifically the present invention describes novel combination products of (i) favipiravir, and (ii) IMPDH inhibitors and their use in therapy, in particular in the treatment of infections caused by RNA viruses.

Combination product can comprise a mixture of (i) inhibitor of viral RNA replication and (ii) IMPDH inhibitor, or a mixture of pharmaceutically acceptable salt, hydrate, solvate, prodrug, stereoisomer, or tautomer thereof.

More specifically combination product can comprise a mixture of (i) favipiravir and (ii) IMPDH inhibitor described herein, or a mixture of pharmaceutically acceptable salt, hydrate, solvate, prodrug, stereoisomer, or tautomer thereof.

In some aspects the combination product is a kit of parts comprising components (i) and (ii), for sequential, separate, or simultaneous use.

In some aspects the combination product is a composition comprising components (i) and (ii) and a pharmaceutically acceptable carrier. In this aspect of the invention pharmaceutical composition comprising combination product and a pharmaceutically acceptable carrier. The pharmaceutical acceptable carrier may further include an excipient, diluent, or surfactant.

Another aspect of the invention relates to a method of treating a disease or disorder associated with RNA virus. The method comprises administering to a patient in need of a treatment for diseases or disorders associated with RNA virus infection an effective amount of a combination product or a pharmaceutical composition described herein.

Another aspect of the invention relates to a method of treating a disease or disorder associated with SARS-CoV-2 virus. The method comprises administering to a patient in need of a treatment for diseases or disorders associated with SARS-CoV-2 virus an effective amount of a combination product or pharmaceutical composition described herein.

Another aspect of the invention is directed to a method of inhibiting RNA virus replication by inhibiting purine biosynthesis. The method involves administering to a patient in need thereof an effective amount of a combination product or a pharmaceutical composition described herein.

Another aspect of the invention is directed to a method of inhibiting SARS-CoV-2 virus replication coupled with inhibiting purine biosynthesis. The method involves administering to a patient in need thereof an effective amount of a combination product or a pharmaceutical composition described herein.

Another aspect of the present invention relates to a combination product of favipiravir and Inosine Monophosphate Nucleotide Dehydrogenase (IMPDH) inhibitor, for use in the manufacture of a medicament for inhibiting of RNA virus replication.

Another aspect of the present invention relates to a combination product of favipiravir and Inosine Monophosphate Nucleotide Dehydrogenase (IMPDH) inhibitor, for use in the manufacture of a medicament for inhibiting of SARS-CoV-2 virus replication.

In a particular embodiment, the combination product of the invention further comprises at least one other therapeutically active agent such as an antifibrotic agent, an anti-inflammatory agent or an immunosuppressive agent.

Another aspect of the present invention relates to a combination product, or a pharmaceutical composition described herein, for use in the manufacture of a medicament for treating or preventing a disease or disorder disclosed herein.

Another aspect of the present invention relates to a combination product, or a pharmaceutical composition described herein, for use in the manufacture of a medicament for treating or preventing a viral infection disclosed herein.

Another aspect of the invention is directed to a method of treating or preventing a disease or disorder disclosed herein in a subject in need thereof. The method involves administering to a patient in need of the treatment an effective amount of a combination product or a pharmaceutical composition disclosed herein.

Another aspect of the present invention relates to the use of a combination product or a pharmaceutical composition thereof, in the treatment of a disease or disorder disclosed herein.

The present invention further provides methods of treating a disease or disorder associated with RNA viral infection, comprising administering to a patient suffering from at least one of said RNA viral infection a combination product or a pharmaceutical composition thereof.

The present invention provides a combination product which acts as inhibitor of RNA virus replication and purine biosynthesis that are therapeutic agents in the treatment of diseases and disorders.

The present invention further provides a combination product and a pharmaceutical composition with an improved efficacy and safety profile relative to known inhibitors of RNA virus replication.

The present invention further provides methods of treating a disease or disorder associated with viral infection, comprising administering to a patient suffering from at least one of said viral infection a combination product or pharmaceutical composition thereof.

The present invention provides combined inhibitors of viral RNA replication and purine biosynthesis that are therapeutic agents in the treatment of viral infections associated with RNA viruses.

The present invention provides combined inhibitors of viral RNA replication and purine biosynthesis that are therapeutic agents in the treatment of viral infections.

The present invention further provides a combination product and a pharmaceutical composition with an improved efficacy and safety profile relative to known viral RNA replication inhibitors. The present disclosure also provides agents with novel mechanisms of action toward viral RNA replication in the treatment of various types of viral infections, including SARS-CoV-2 infection.

Another aspect of the invention relates to lowering the normal dose of favipiravir needed for effective antiviral therapy.

Another aspect of the invention relates to lowering the normal dose of favipiravir needed for effective SARS-CoV-2 therapy.

Another aspect of the invention relates to lowering the dose of Inosine Monophosphate Nucleotide Dehydrogenase (IMPDH) inhibitor needed to increase inhibition of viral RNA replication.

Another aspect of the invention relates to lowering the dose of Inosine Monophosphate Nucleotide Dehydrogenase (IMPDH) inhibitor needed to increase inhibition of SARS-CoV-2 virus replication.

Another aspect of the invention relates to lowering the dose of mycophenolate mofetil needed to increase inhibition of viral RNA replication.

Another aspect of the invention relates to lowering the dose of mycophenolate mofetil needed to increase inhibition of SARS-CoV-2 virus replication.

The present invention further provides methods of preventing, treating, or ameliorating a viral infection caused by virus selected from Measles, Mumps, Respiratory Syncytial Virus, Metapneumovirus, Poliovirus, Chikungunya virus, Hepatitis A virus, Hepatitis C virus, Hepatitis E virus, West Nile virus, Zika virus, Dengue virus, Lassa Fever virus, Junin South American hemorrhagic fever virus, Marburg virus, Ebola virus, Norovirus (human caliciviruses) infections including the Norwalk agent, Rift Valley Fever virus, Nipah virus, Hendra virus, HIV-1, HTLV-1, HTLV-2, rotavirus, seasonal and pandemic coronaviruses, Picobirnaviruses, Toroviruses, and Astroviruses.

The present invention further provides methods of treating, or ameliorating a viral infection caused by virus selected from Measles, Mumps, Respiratory Syncytial Virus, Metapneumovirus, Poliovirus, Chikungunya virus, Hepatitis A virus, Hepatitis C virus, Hepatitis E virus, West Nile virus, Zika virus, Dengue virus, Lassa Fever virus, Junin South American hemorrhagic fever virus, Marburg virus, Ebola virus, Norovirus (human caliciviruses) infections including the Norwalk agent, Rift Valley Fever virus, Nipah virus, Hendra virus, HIV-1, HTLV-1, HTLV-2, rotavirus, seasonal and pandemic coronaviruses, Picobirnaviruses, Toroviruses, and Astroviruses comprising administering to a patient suffering from at least one of said viral infection a combination product or pharmaceutical composition thereof.

In some aspects, the present disclosure provides a method of preparing a combination product or a pharmaceutical composition of the present disclosure.

In some aspects, the present disclosure provides a method of preparing combination product of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims

### BRIEF DESCRIPTION OF THE DRAWINGS

FIG. 1: Concentration-effect relationship for the inhibition (%) of influenza virus cytopathic activity for Favipiravir and Mycophenolate. MDCK cells were pre-treated for 1 h with two-fold serial dilutions of Favipiravir (A) or Mycophenolate (B). Cells were infected with influenza (strain A/PR8) at m.o.i. 0.01. After removing the virus inoculum, the cells were further maintained in medium containing drugs for two days. For each compound, activity was expressed relative to uninfected/untreated controls (100% inhibition of viral cytopathic activity) and infected/untreated controls (0% inhibition of viral activity). For Favipiravir, activity was assessed at concentrations ranging from 100 μM to 0.78 μM with two-fold serial dilutions in triplicate. For Mycophenolate, activity was assessed at concentrations ranging from 2 μM to 0.25 μM with two-fold serial dilutions in triplicate. IC₅₀ for each compound was calculated using a four-parameter logistic nonlinear regression model in GraphPad Prism and is indicated for each drug. Cell viability at each level relative to control is shown (triangles).

FIG. 2 and FIG. 3: Concentration-effect relationship for the inhibition (%) of influenza virus cytopathic activity for Favipiravir and Mycophenolate combinations. MDCK cells were pre-treated for 1 h. with two-fold serial dilutions of Favipiravir in the presence of several fixed concentrations of Mycophenolate. The cells were infected with influenza at m.o.i. 0.01. After removing the virus inoculum, the cells were further maintained in the medium containing drugs for two days. For each drug combination, activity was expressed relative to uninfected/untreated controls (100% inhibition of viral cytopathic activity) and infected/untreated controls (0% inhibition of viral activity). IC₅₀ for each drug combination was calculated using a four-parameter logistic nonlinear regression model in GraphPad Prism and is indicated. Note the IC₅₀ of Favipiravir alone (FIG. 1) is 26.5 μM and the IC₅₀ for Mycophenolate alone was 1.4 μM.

FIG. 4, FIG. 5, and FIG. 6. Favipiravir and Mycophenolate display synergistic interaction at specific concentrations. The Synergy Finder 2.0 was used to calculate the highest single agent (HSA) synergy score of two-drug combinations from different pairwise combinations. The dose-response matrix (FIG. 4) and the synergy map of two-drug combinations treatment (FIG. 5 and FIG. 6) are shown. Based on the “Most synergistic area score” (FIG. 5 and FIG. 6), the combination of Favipiravir at concentrations ranges from 6.2 μM to 25 μM and Mycophenolate at concentrations range from 0.2 μM to 1 μM might display best synergistic interaction (FIG. 5 and FIG. 6). Areas with synergy score less than −10: the interaction between two drugs is likely to be antagonistic: from −10 to 10: the interaction between two drugs is likely to be additive: larger than 10: the interaction between two drugs is likely to be synergistic.

FIG. 7. Concentration-effect relationship for the inhibition (%) of influenza virus cytopathic activity for Favipiravir and Viramidine. MDCK cells were pre-treated for 1 hr. with two-fold serial dilutions of Favipiravir or Viramidine. The cells were infected with influenza at m.o.i. 0.01. After removing the virus inoculum, the cells were further maintained in the medium containing drugs for two days. For each compound, activity was expressed relative to uninfected/untreated controls (100% inhibition of viral cytopathic activity) and infected/untreated controls (0% inhibition of viral activity). For Favipiravir, activity was assessed at concentrations range from 100 μM to 0.78 μM with two-fold serial dilutions in triplicate. For Mycophenolate, activity was assessed at concentrations range from 200 μM to 1.56 μM with two-fold serial dilutions in triplicate. IC₅₀ for each compound was calculated using a four-parameter logistic nonlinear regression model in GraphPad Prism and is indicated. Cell viability at each level relative to control is shown (triangles).

FIG. 8 and FIG. 9. Concentration-effect relationship for the inhibition (%) of influenza virus cytopathic activity for Favipiravir and Viramidine combinations. MDCK cells were pre-treated for 1 h. with two-fold serial dilutions of Favipiravir in the presence of different fixed concentrations of Viramidine. The cells were infected with influenza at m.o.i. 0.01. After removing the virus inoculum, the cells were further maintained in medium containing drugs for two days. For each drug combination, activity was expressed relative to uninfected/untreated controls (100% inhibition of viral cytopathic activity) and infected/untreated controls (0% inhibition of viral activity). IC₅₀ for each drug combination was calculated using a four-parameter logistic nonlinear regression model in GraphPad Prism and is indicated. Note that the IC₅₀ for Favipiravir alone (FIG. 7) was 25.13 μM for this experiment and the IC₅₀ for Viramidine alone was 94.7 μM.

FIG. 10, FIG. 11, and FIG. 12. Favipiravir and Viramidine display synergistic interaction at specific concentrations. The Synergy Finder2.0 was used to calculate the highest single agent (HSA) synergy score of two-drug combinations from different pairwise combinations. The dose-response matrix (FIG. 10) and the synergy map of two-drug combinations treatment (FIG. 11 and FIG. 12) are shown. Based on the “Most synergistic area score” (FIG. 11 and FIG. 12), the combination of Favipiravir at concentrations range from 6.2 μM to 25 μM and Viramidine at concentrations range from 25 μM to 100 μM might display best synergistic interaction (FIG. 11 and FIG. 12). Areas with synergy score less than −10: the interaction between two drugs is likely to be antagonistic: from −10 to 10: the interaction between two drugs is likely to be additive: larger than 10: the interaction between two drugs is likely to be synergistic.

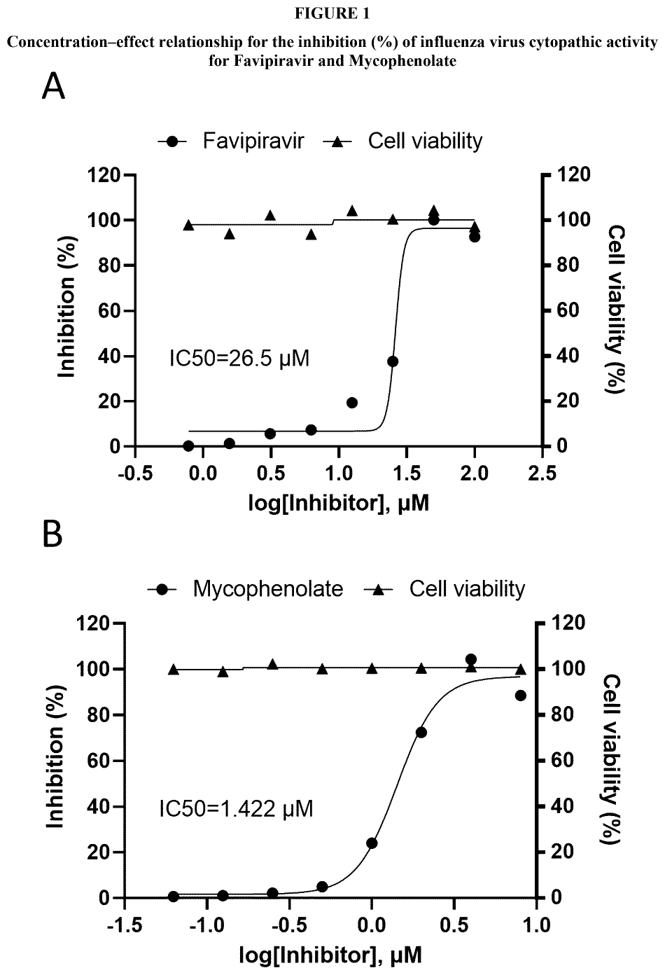

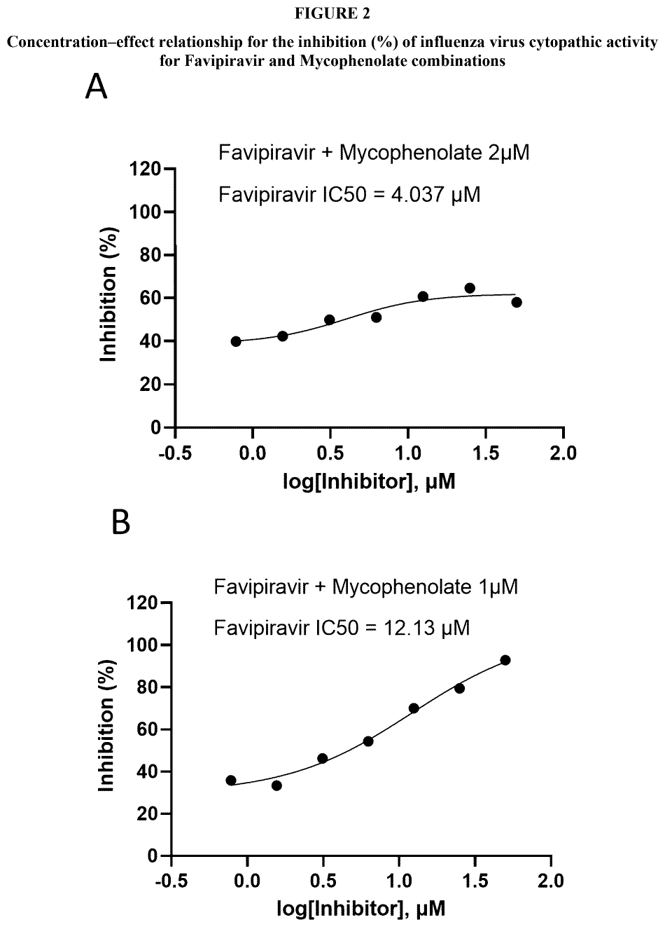

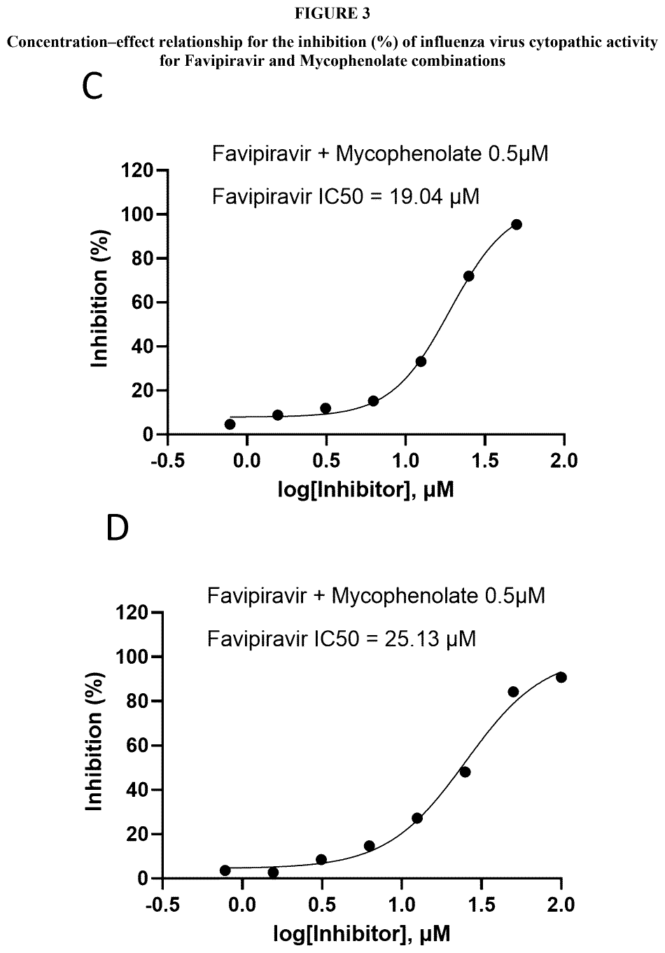

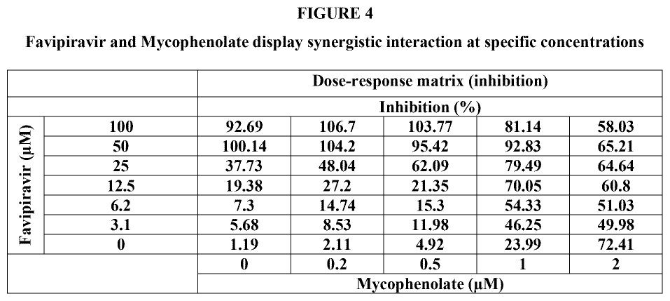

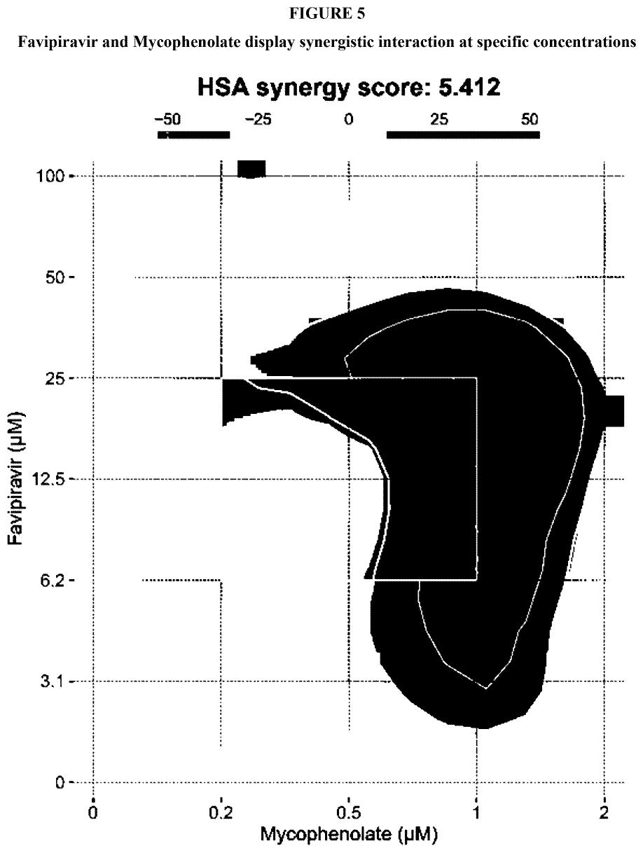

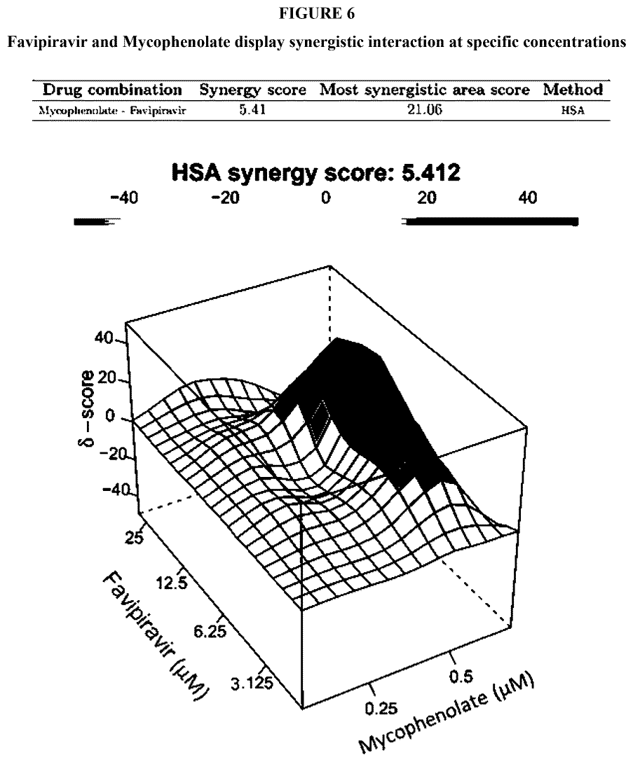

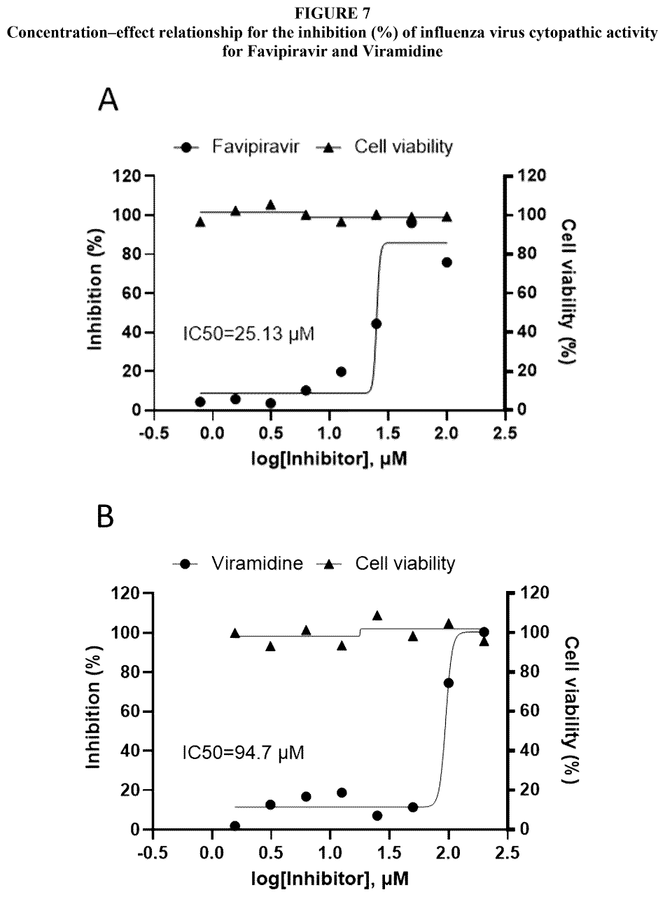

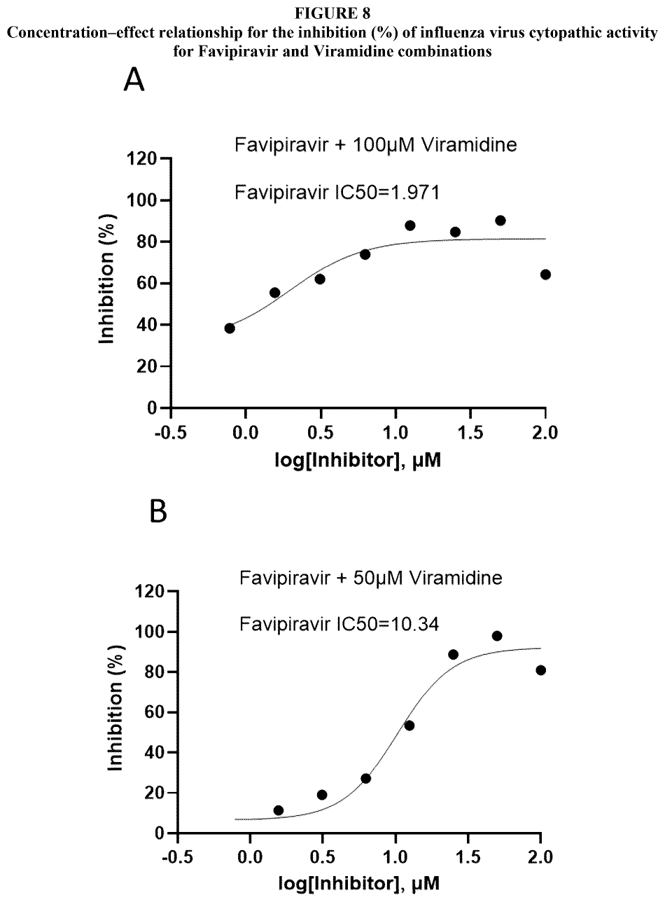

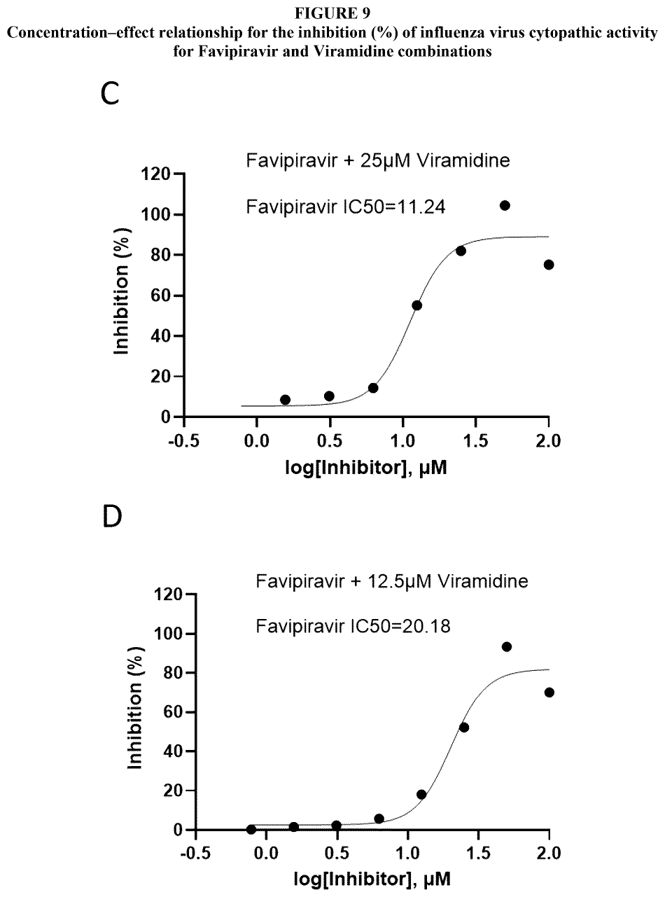

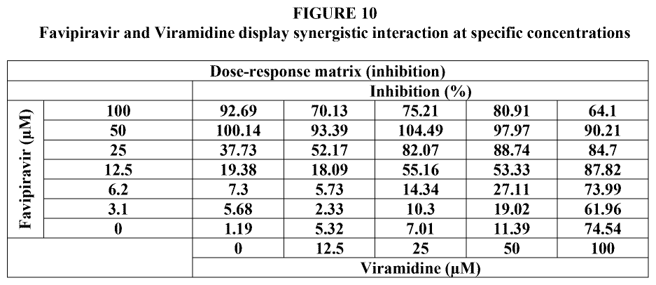

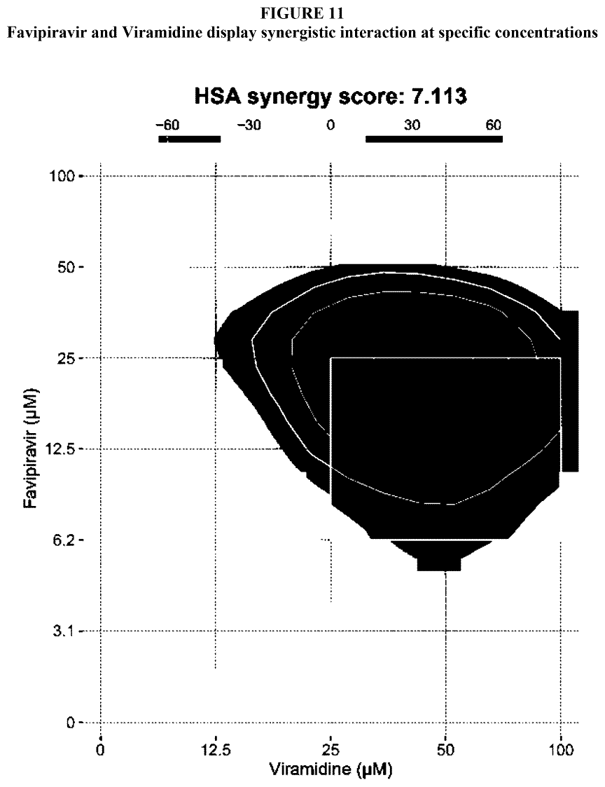

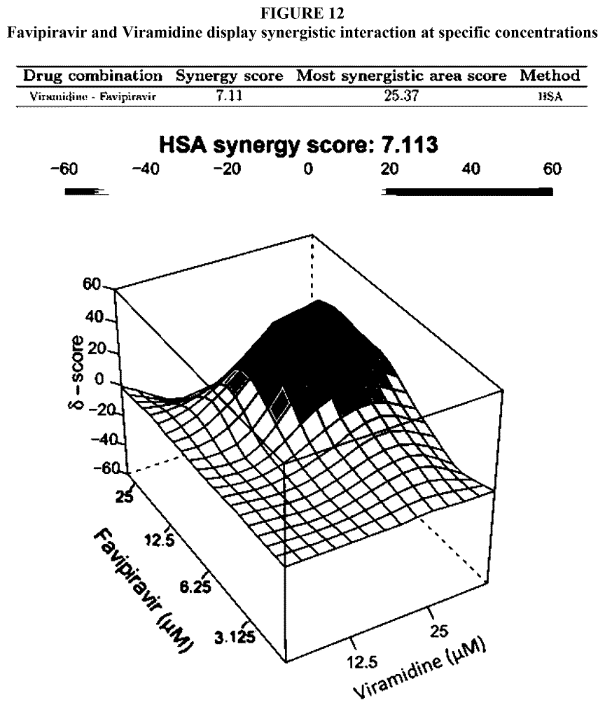

### DETAILED DESCRIPTION

The present disclosure provides methods of treating, preventing, or ameliorating a disease or disorder associated with RNA virus infection by administering to a subject in need thereof a therapeutically effective amount of a combination product as disclosed herein.

The present invention provides a combination of an inhibitor viral RNA replication with IMPDH inhibitors with improved activity profile and lower dose of each compound and toxicity.

The inventors found that favipiravir in combination with IMPDH inhibitors show therapeutic activities that are useful in therapy, in particular for the treatment of infections caused by RNA viruses.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “combination” and/or “combination product” refers to a product composed of any combination of a drug. Each drug included in a combination product is referred to as a “constituent part” of the combination product. The combination product is a kit of parts comprising components i) and ii), for sequential, separate, or simultaneous use or a composition (a mixture) of two components i) and ii).

The term “pharmaceutical composition” refers to admixture of different chemical substances, including the active drug (or number of active drugs), are combined to produce a final medicinal product.

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents, and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

A “viral disease” (or “viral infection”, abbreviated vid or VID) occurs when an organism's body is invaded by pathogenic viruses, and infectious virus particles (virions) attach to and enter susceptible cells.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed combination product or a composition to a subject, or administering a prodrug derivative or analog of the compounds of combination product or composition to the subject, which can form an equivalent amount of active compound within the subject's body.

The term “prodrug”, as used in this disclosure, means a compound which is convertible in vivo by metabolic means (e.g., by hydrolysis) to a disclosed compound.

The term “salt’ refers to pharmaceutically acceptable salts.

“Inhibitors RNA virus replication” as used herein refer to combination product and/or compositions comprising a combination product which inhibit of viral RNA replication.

The amount of combination product or composition described herein needed for achieving a therapeutic effect may be determined empirically in accordance with conventional procedures for the particular purpose. Generally, for administering therapeutic agents (e.g. combination products or compositions (and/or additional agents) described herein) for therapeutic purposes, the therapeutic agents are given at a pharmacologically effective dose.

A “pharmacologically effective amount”, “pharmacologically effective dose”, “therapeutically effective amount”, or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from SARS-CoV-2 infection provides a therapeutic benefit not only when the underlying condition is eradicated or ameliorated, but also when the subject reports a decrease in the severity or duration of the symptoms associated with the disease, e.g., a decrease in viral burden, a decrease in circulating of SARS-CoV-2 viruses, an increase in progression free survival. Therapeutic benefit also includes halting or slowing the progression of the underlying disease or disorder, regardless of whether improvement is realized.

### Combination Product of the Present Disclosure

A first aspect of the invention relates to a combination product of (i) inhibitor viral RNA replication and (ii) Inosine Monophosphate Nucleotide Dehydrogenase (IMPDH) inhibitor.

More specifically, the present disclosure provides a combination therapy for RNA viral infection treatment by administering of favipiravir and Inosine Monophosphate Nucleotide Dehydrogenase (IMPDH) inhibitor.

In preferred embodiments, the combination product according to invention comprises:
(i) favipiravir and
(ii) IMPDH inhibitor.

In some embodiments, the combination product comprises favipiravir (i) as a nucleoside base (favipiravir), favipiravir ribonucleotide or favipiravir nucleotide triphosphate (favipiravir-NTP):

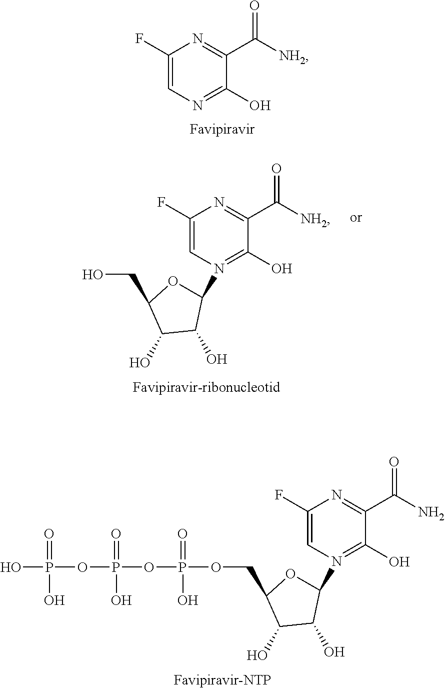

or a pharmaceutically acceptable salt, solvate, or tautomer thereof.

Favipiravir, a purine nucleoside analogue capable of causing lethal mutagenesis during RNA virus replication.

The pathway for purine biosynthesis, indicating the crucial role for Inosine Monophosphate Nucleotide Dehydrogenase (IMPDH). IMPDH was selected as the preferred target for drugs to pair with favipiravir due to the availability of many generic drugs with known mechanisms of action and low toxicity.

The mechanism of action of IMPDH inhibitor presented at the scheme below:

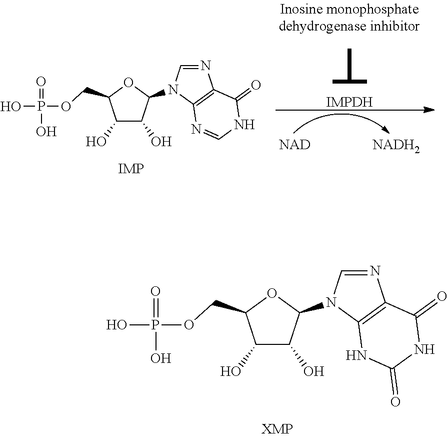

In some embodiments, the IMPDH inhibitor (ii) is selected from:

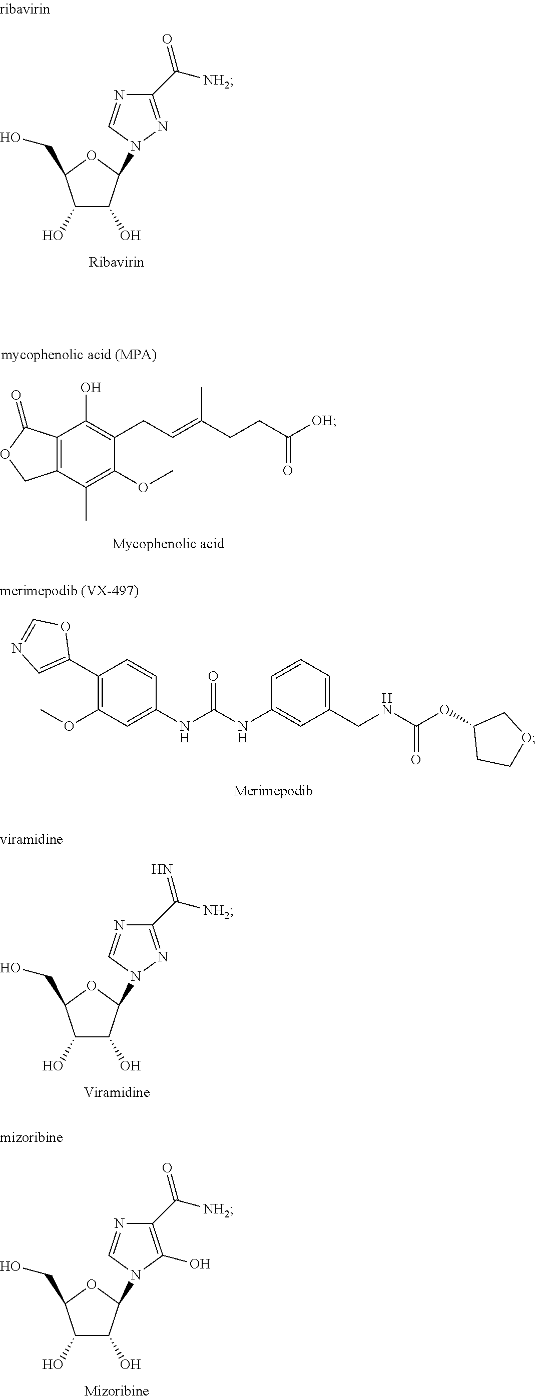 [SMILES: NC(=O)c1ncn([C@@H]2O[C@H](CO)[C@@H](O)[C@H]2O)n1 ; COc1c(C)c2c(c(O)c1C/C=C(\C)CCC(=O)O)C(=O)OC2 ; COc1cc(NC(=O)Nc2cccc(CNC(=O)O[C@H]3CCOC3)c2)ccc1-c1cnco1 ; N=C(N)c1ncn([C@@H]2O[C@H](CO)[C@@H](O)[C@H]2O)n1 ; NC(=O)c1ncn([C@@H]2O[C@H](CO)[C@@H](O)[C@H]2O)c1O]

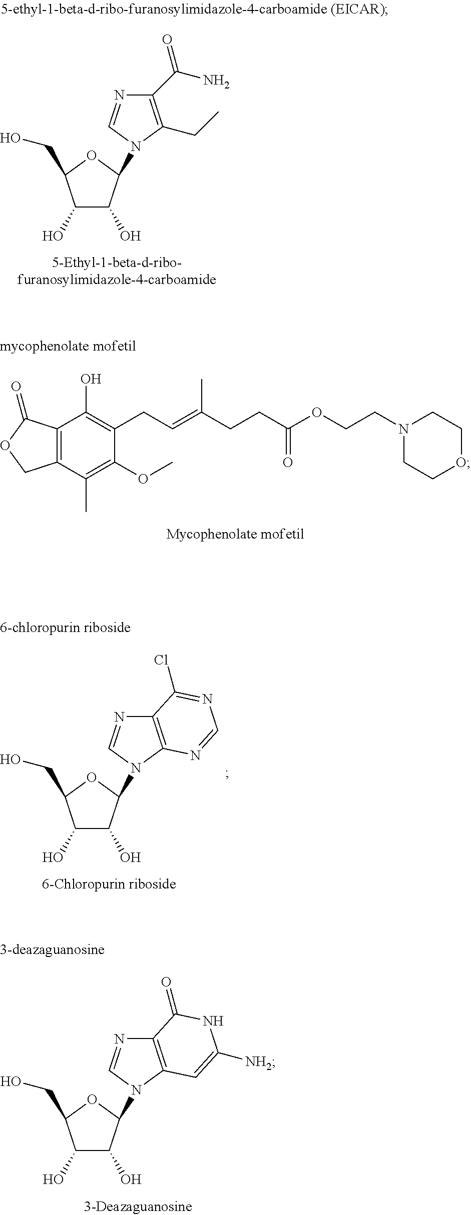 [SMILES: CCc1c(C(N)=O)ncn1[C@@H]1O[C@H](CO)[C@@H](O)[C@H]1O ; COc1c(C)c2c(c(O)c1C/C=C(\C)CCC(=O)OCCN1CCOCC1)C(=O)OC2 ; OC[C@H]1O[C@@H](n2cnc3c(Cl)ncnc32)[C@H](O)[C@@H]1O ; Nc1cc2c(ncn2[C@@H]2O[C@H](CO)[C@@H](O)[C@H]2O)c(=O)[nH]1]

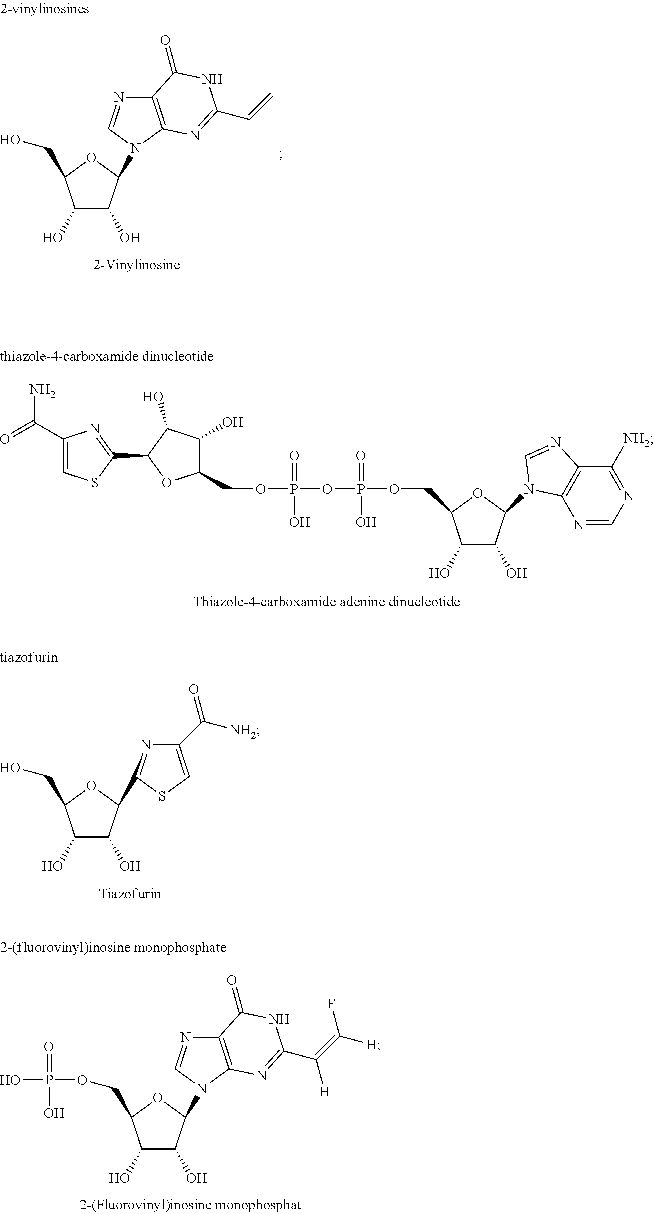

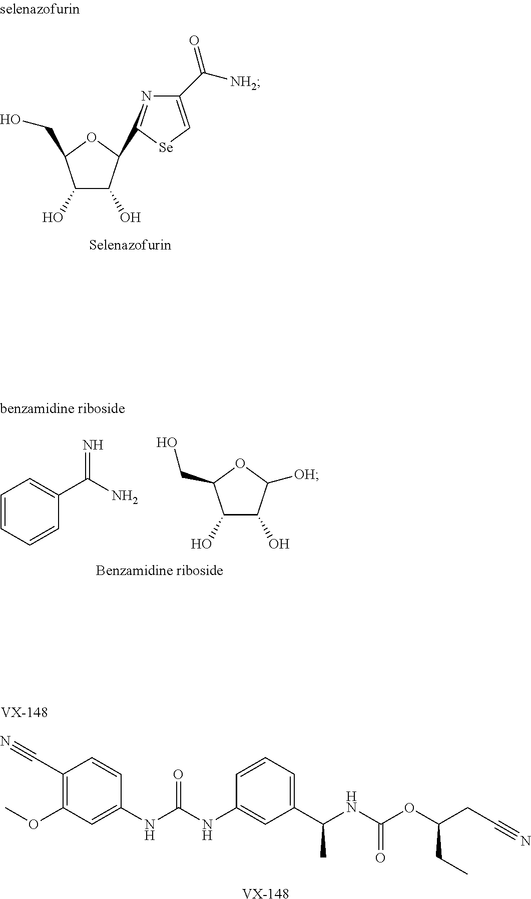

or a pharmaceutically acceptable salt, stereoisomer, solvate, or tautomer thereof.

In some embodiments, a combination product comprises (i) favipiravir and (ii) ribavirin.

In some embodiments, a combination product comprises (i) favipiravir and (ii) mycophenolic acid (MPA).

In some embodiments, a combination product comprises (i) favipiravir and (ii) merimepodib (VX-497).

In some embodiments, a combination product comprises (i) favipiravir and (ii) viramidine.

In some embodiments, a combination product comprises (i) favipiravir and (ii) mizoribine.

In some embodiments, a combination product comprises (i) favipiravir and (ii) 5-ethyl-1-beta-d-ribo-furanosylimidazole-4-carboamide (EICAR).

In some embodiments, a combination product comprises (i) favipiravir and (ii) mycophenolate mofetil.

In some embodiments, a combination product comprises (i) favipiravir and (ii) 6-chloropurin riboside.

In some embodiments, a combination product comprises (i) favipiravir and (ii) 3-deazaguanosine.

In some embodiments, a combination product comprises (i) favipiravir and (ii) 2-vinylinosines.

In some embodiments, a combination product comprises (i) favipiravir and (ii) thiazole-4-carboxamide dinucleotide.

In some embodiments, a combination product comprises (i) favipiravir and (ii) tiazofurin.

In some embodiments, a combination product comprises (i) favipiravir and (ii) 2-(fluorovinyl) inosine monophosphate.

In some embodiments, a combination product comprises (i) favipiravir and (ii) selenazofurin.

In some embodiments, a combination product comprises (i) favipiravir and (ii) benzamidine riboside.

In some embodiments, a combination product comprises (i) favipiravir and (ii) VX-148.

In some embodiments, the combination product is selected from the combinations described in Table 1.

TABLE 1
Examples of composition of combination product.

| | Inhibitor of viral | | |
|---|---|---|---|
| | RNA replication | IMPDH inhibitor | Molar ratio |
| # | (i) | (ii) | (i)/(ii) |
| 1 | favipiravir | ribavirin | 1:100-100:1 |
| 2 | favipiravir | mycophenolic acid (MPA) | 1:100-100:1 |
| 3 | favipiravir | merimepodib (VX-497) | 1:100-100:1 |
| 4 | favipiravir | viramidine | 1:100-100:1 |
| 5 | favipiravir | mizoribine | 1:100-100:1 |
| 6 | favipiravir | 5-ethyl-1-beta-d-ribo- | 1:100-100:1 |
| | | furanosylimidazole-4- | |
| | | carboamide (EICAR) | |
| 7 | favipiravir | mycophenolate mofetil | 1:100-100:1 |
| 8 | favipiravir | 6-chloropurin riboside | 1:100-100:1 |
| 9 | favipiravir | 3-deazaguanosine | 1:100-100:1 |
| 10 | favipiravir | 2-vinylinosines | 1:100-100:1 |
| 11 | favipiravir | thiazole-4-carboxamide | 1:100-100:1 |
| | | dinucleotide | |
| 12 | favipiravir | tiazofurin | 1:100-100:1 |
| 13 | favipiravir | 2-(fluorovinyl)inosine | 1:100-100:1 |
| | | monophosphate | |
| 14 | favipiravir | selenazofurin | 1:100-100:1 |
| 15 | favipiravir | benzamidine riboside | 1:100-100:1 |
| 16 | favipiravir | VX-148 | 1:100-100:1 |
| 17 | favipiravir | ribavirin | 1:100-100:1 |
| | ribonucleotide | | |
| 18 | favipiravir | mycophenolic acid (MPA) | 1:100-100:1 |
| | ribonucleotide | | |
| 19 | favipiravir | merimepodib (VX-497) | 1:100-100:1 |
| | ribonucleotide | | |
| 20 | favipiravir | viramidine | 1:100-100:1 |
| | ribonucleotide | | |
| 21 | favipiravir | mizoribine | 1:100-100:1 |
| | ribonucleotide | | |
| 22 | favipiravir | 5-ethyl-1-beta-d-ribo- | 1:100-100:1 |
| | ribonucleotide | furanosylimidazole-4- | |
| | | carboamide (EICAR) | |
| 23 | favipiravir | mycophenolate mofetil | 1:100-100:1 |
| | ribonucleotide | | |
| 24 | favipiravir | 6-chloropurin riboside | 1:100-100:1 |
| | ribonucleotide | | |
| 25 | favipiravir | 3-deazaguanosine | 1:100-100:1 |
| | ribonucleotide | | |
| 26 | favipiravir | 2-vinylinosines | 1:100-100:1 |
| | ribonucleotide | | |
| 27 | favipiravir | thiazole-4-carboxamide | 1:100-100:1 |
| | ribonucleotide | dinucleotide | |
| 28 | favipiravir | tiazofurin | 1:100-100:1 |
| | ribonucleotide | | |
| 29 | favipiravir | 2-(fluorovinyl)inosine | 1:100-100:1 |
| | ribonucleotide | monophosphate | |
| 30 | favipiravir | selenazofurin | 1:100-100:1 |
| | ribonucleotide | | |
| 31 | favipiravir | benzamidine riboside | 1:100-100:1 |
| | ribonucleotide | | |
| 32 | favipiravir | VX-148 | 1:100-100:1 |
| | ribonucleotide | | |
| 33 | favipiravir-NTF | ribavirin | 1:100-100:1 |
| 34 | favipiravir-NTF | mycophenolic acid (MPA) | 1:100-100:1 |
| 35 | favipiravir-NTF | merimepodib (VX-497) | 1:100-100:1 |
| 36 | favipiravir-NTF | viramidine | 1:100-100:1 |
| 37 | favipiravir-NTF | mizoribine | 1:100-100:1 |
| 38 | favipiravir-NTF | 5-ethyl-1-beta-d-ribo- | 1:100-100:1 |
| | | furanosylimidazole-4- | |
| | | carboamide (EICAR) | |
| 39 | favipiravir-NTF | mycophenolate mofetil | 1:100-100:1 |
| 40 | favipiravir-NTF | 6-chloropurin riboside | 1:100-100:1 |
| 41 | favipiravir-NTF | 3-deazaguanosine | 1:100-100:1 |
| 42 | favipiravir-NTF | 2-vinylinosines | 1:100-100:1 |
| 43 | favipiravir-NTF | thiazole-4-carboxamide | 1:100-100:1 |
| | | dinucleotide | |
| 44 | favipiravir-NTF | tiazofurin | 1:100-100:1 |
| 45 | favipiravir-NTF | 2-(fluorovinyl)inosine | 1:100-100:1 |
| | | monophosphate | |
| 46 | favipiravir-NTF | selenazofurin | 1:100-100:1 |
| 47 | favipiravir-NTF | benzamidine riboside | 1:100-100:1 |
| 48 | favipiravir-NTF | VX-148 | 1:100-100:1 |

In some embodiments, compounds formed combination product described herein are pharmaceutically acceptable salts, stereoisomers, solvates, prodrugs, isotopic derivatives, or tautomers of these compounds.

A suitable pharmaceutically acceptable salt of a compound of the combination is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It would be understood that the compounds of any one of the combination products disclosed herein and any pharmaceutically acceptable salts thereof, comprise stereoisomers, mixtures of stereoisomers, polymorphs of all isomeric forms of said compounds.

In some embodiments, the compounds of the combination product are selected from the compounds described in Table 1 and pharmaceutically acceptable salts, stereoisomers, solvates, prodrugs, isotopic derivatives, or tautomers thereof.

In some embodiments, the compounds of the combination product are selected from the compounds described in Table 1 and prodrugs and pharmaceutically acceptable salts thereof.

In some embodiments, the compounds of the combination product are selected from the compounds described in Table 1 and pharmaceutically acceptable salts thereof.

In some embodiments, the compounds of the combination product are selected from the prodrugs of the compounds described in Table 1 and pharmaceutically acceptable salts thereof.

In some embodiments, the compounds of the combination product are selected from the compounds described in Table 1.

In some embodiments, the compound of the combination product is a pharmaceutically acceptable salt of any one of the compounds described in Table 1.

In some embodiments, the compound of the combination product is a lithium salt, sodium salt, potassium salt, calcium salt, or magnesium salt of any one of the compounds described in Table 1.

In some embodiments, the compound of the combination product is a sodium salt or potassium salt of any one of the compounds described in Table 1.

In some embodiments, the compound of the combination product is a salt of any acid described in the Table 2 and any one of the compounds described in Table 1.

TABLE 2
Pharmaceutical acceptable acid forming salts with
the compounds of the combination product.

1-hydroxy-2-naphthoic acid
2,2-dichloroacetic acid
2-hydroxyethanesulfonic acid
2-oxoglutaric acid
4-acetamidobenzoic acid
4-aminosalicylic acid
acetic acid
adipic acid
ascorbic acid (L)
aspartic acid (L)
benzenesulfonic acid
benzoic acid
camphoric acid (+)
camphor-10-sulfonic acid (+)
capric acid (decanoic acid)
caproic acid (hexanoic acid)
caprylic acid (octanoic acid)
carbonic acid
cinnamic acid
citric acid
cyclamic acid
dodecylsulfuric acid
ethane-1,2-disulfonic acid
ethanesulfonic acid
formic acid
fumaric acid
galactaric acid
gentisic acid
glucoheptonic acid (D)
gluconic acid (D)
glucuronic acid (D)
glutamic acid
glutaric acid
glycerophosphoric acid
glycolic acid
hippuric acid
hydrobromic acid
hydrochloric acid
isobutyric acid
lactic acid (DL)
lactobionic acid
lauric acid
maleic acid
malic acid (−L)
malonic acid
mandelic acid (DL)
methanesulfonic acid
naphthalene-1,5-disulfonic acid
naphthalene-2-sulfonic acid
nicotinic acid
nitric acid
oleic acid
oxalic acid
palmitic acid
pamoic acid
phosphoric acid
proprionic acid
pyroglutamic acid (−L)
salicylic acid
sebacic acid
stearic acid
succinic acid
sulfuric acid
tartaric acid (+L)
thiocyanic acid
toluenesulfonic acid (p)
undecylenic acid

In some aspects, the present disclosure provides a compound of the combination product being an isotopic derivative (e.g., isotopically labeled compound) of any one of the compounds disclosed herein.

In some embodiments, the compound is an isotopic derivative of any one of the compounds described in Table 1 and prodrugs and pharmaceutically acceptable salts thereof.

In some embodiments, at least one compound of combination product is an isotopic derivative of any one of the compounds described in Table 1 and pharmaceutically acceptable salts thereof.

In some embodiments, at least one compound of combination product is an isotopic derivative of any one of prodrugs of the compounds described in Table 1 and pharmaceutically acceptable salts thereof.

In some embodiments, at least one compound of combination product is an isotopic derivative of any one of the compounds described in Table 1.

In some embodiments, the isotopic derivative is a deuterium labeled compound.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof).

In some embodiments, at least one compound of combination product is a deuterium labeled compound of any one of the compounds described in Table 1 and prodrugs and pharmaceutically acceptable salts thereof.

In some embodiments, at least one compound of combination product is a deuterium labeled compound of any one of the compounds described in Table 1 and pharmaceutically acceptable salts thereof.

In some embodiments, at least one compound of combination product is a deuterium labeled compound of any one of the prodrugs of the compounds described in Table 1 and pharmaceutically acceptable salts thereof.

In some embodiments, at least one compound of combination product is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

In some embodiments, at least one compound of combination product is a ¹⁸F labeled compound.

In some embodiments, at least one compound of combination product is a ³³S labeled compound, a ³⁴S labeled compound, a ³⁵S labeled compound, a ³⁶S labeled compound, or any combination thereof.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of combination product of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of combination product of this disclosure may possess one or more asymmetric centres: such compounds can therefore be produced as individual (R)- or(S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

It is also to be understood that certain compounds of any one of combination product disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the combination products disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the combination products disclosed herein may exist in a number of different tautomeric forms and references to compound of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

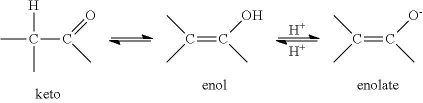

The compounds of any one of the combination products disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the compounds of combination products disclosed herein.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985): b) Design of Pro-drugs, edited by H. Bundgaard, (Elsevier, 1985): c) A Textbook of Drug Design and Development, edited by Krogsgaard-Larsen and H. Bundgaard, Chapter 5 “Design and Application of Pro-drugs”, by H. Bundgaard p. 113-191 (1991): d) H. Bundgaard, Advanced Drug Delivery Reviews, 8, 1-38 (1992): e) H. Bundgaard, et al., Journal of Pharmaceutical Sciences, 77, 285 (1988): f) N. Kakeya, et al., Chem. Pharm. Bull., 32, 692 (1984): g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the combination products disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the combination products disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the combination products disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl.

In some embodiments, the combination product is a combination of a pharmaceutical composition of component (i) and a pharmaceutical composition of component (ii).

In some embodiments, the combination product is a combination of pharmaceutical composition of favipiravir (i) and a pharmaceutical composition of IMPDH inhibitor (ii).

In some embodiments, the combination product is a kit of parts comprising components (i) and (ii), for sequential, separate, or simultaneous use.

In some embodiments, the combination product is a kit of parts comprising components (i) and (ii) for sequential use.

In some embodiments, the combination product is a kit of parts comprising components (i) and (ii) for separate use.

In some embodiments, the combination product is a kit of parts comprising components (i) and (ii) for simultaneous use.

In some embodiments, the combination product is a composition comprising components (i) and (ii) and a pharmaceutically acceptable carrier.

The in vivo effects of a compound of any one of the combination products disclosed herein may be exerted in part by one or more metabolites that are formed within the human or animal body after administration of a compound of any one of the combination product disclosed herein. As stated hereinbefore, the in vivo effects of a compound of any one of the combination products disclosed herein may also be exerted by way of metabolism of a precursor compound (a prodrug).

### Pharmaceutical Compositions

In some aspects, the present disclosure provides a pharmaceutical composition comprising the compounds of the combination product as active ingredients. In some embodiments, the present disclosure provides a pharmaceutical composition comprising at least one inhibitor of viral RNA replication (i) and at least one IMPDH inhibitor (ii), or their pharmaceutically acceptable salt or solvate, and one or more pharmaceutically acceptable carriers or excipients.

In some embodiments, the present disclosure provides a pharmaceutical composition comprising (i) Favipiravir or pharmaceutically acceptable salt, solvate, or tautomer thereof: (ii) IMPDH inhibitor or pharmaceutically acceptable salt, solvate, or tautomer thereof and a pharmaceutically acceptable carrier.

In some embodiments, the present disclosure provides a pharmaceutical composition comprising inhibitor of viral RNA replication and at least one IMPDH inhibitor (ii) selected from the Table 1.

In some embodiments, a pharmaceutical composition comprising (i) favipiravir or pharmaceutically acceptable salt, solvate, or tautomer thereof: (ii) ribavirin or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) mycophenolic acid (MPA) or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) merimepodib (VX-497) or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) viramidine or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) mizoribine or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) 5-ethyl-1-beta-d-ribo-furanosylimidazole-4-carboamide (EICAR) or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) mycophenolate mofetil or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) 6-chloropurin riboside or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) 3-deazaguanosine or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) 2-vinylinosines or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) thiazole-4-carboxamide dinucleotide or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) tiazofurin or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) 2-(fluorovinyl) inosine monophosphate or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) selenazofurin or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) benzamidine riboside or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof: (ii) VX-148 or pharmaceutically acceptable salt, solvate, or tautomer thereof and a pharmaceutically acceptable carrier.

As used herein, the term “pharmaceutical composition” is intended to encompass a product comprising the specified ingredients in the specified amounts, as well as any product which results, directly or indirectly, from combination of the specified ingredients in the specified amounts.

The pharmaceutical composition of present disclosure can be formulated for oral administration in forms such as tablets, capsules (each of which includes sustained release or timed-release formulations), pills, powders, granules, elixirs, tinctures, suspensions, syrups, and emulsions.

In some embodiments, a pharmaceutical composition of present disclosure can be formulated for oral administration in form of tablets.

In some embodiments, a pharmaceutical composition of present disclosure can be formulated for oral administration in form of capsules (each of which includes sustained release or timed-release formulations).

In some embodiments, a pharmaceutical composition of present disclosure can be formulated for oral administration in form of pills.

In some embodiments, a pharmaceutical composition of present disclosure can be formulated for oral administration in form of powders.

In some embodiments, a pharmaceutical composition of present disclosure can be formulated for oral administration in form of granules.

In some embodiments, a pharmaceutical composition of present disclosure can be formulated for oral administration in form of elixirs.

In some embodiments, a pharmaceutical composition of present disclosure can be formulated for oral administration in form of tinctures.

In some embodiments, a pharmaceutical composition of present disclosure can be formulated for oral administration in form of suspensions.

In some embodiments, a pharmaceutical composition of present disclosure can be formulated for oral administration in form of syrups.

In some embodiments, a pharmaceutical composition of present disclosure can be formulated for oral administration in form of emulsions.

The pharmaceutical composition of present disclosure can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxy benzoate, propylaminopropyl biguanide, and butyl-p-hydroxy benzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxy benzoate, propylaminopropyl biguanide, and butyl-p-hydroxy benzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and 8-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin: an excipient such as starch or lactose, a disintegrating agent such as alginic acid, Primogel, or corn starch; a lubricant such as magnesium stearate or Sterotes: a glidant such as colloidal silicon dioxide: a sweetening agent such as sucrose or saccharin: or a flavoring agent such as peppermint, methyl salicylate, or orange flavoring.

According to a further aspect of the disclosure there is provided a pharmaceutical composition which comprises compounds (i) and (ii) of a combination product of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The pharmaceutical compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a viral infection referred to herein, slow its progression and/or reduce the symptoms associated with the condition.

A therapeutically effective amount of a compound of the present disclosure for use in therapy is an amount sufficient to treat a viral infection related condition referred to herein, slow its progression and/or reduce the symptoms associated with the condition.

The size of the dose for therapeutic or prophylactic purposes of a combination product will naturally vary according to the nature and severity of the conditions, the age and sex of the animal or subject and the route of administration, according to well-known principles of medicine.

### Biological Assays

Combination product designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the combination products described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the combination products of the present disclosure. These in vitro or in vivo biological assays can include, but are not limited to, enzymatic activity assays, electrophoretic mobility shift assays, reporter gene assays, in vitro cell viability assays, and the assays described herein.

### Methods of Use

In some aspects, the present disclosure provides a method of inhibition of replication of RNA virus (e.g., in vitro or in vivo), comprising contacting a cell with a therapeutically effective amount of a combination product of the present disclosure.

In some aspects, the present disclosure provides a method of treating or preventing a disease or disorder disclosed herein in a subject in need thereof, comprising administering to the subject a therapeutically effective amount of a combination product of the present disclosure or a pharmaceutical composition of the present disclosure.

In some aspects, the present disclosure provides a method of treating a disease or disorder disclosed herein in a subject in need thereof, comprising administering to the subject a therapeutically effective amount of a combination product of the present disclosure or a pharmaceutical composition of the present disclosure.

In some embodiments, the disease or disorder is an RNA viral infection.

In some embodiments, the disease or disorder is a disease or disorder in which RNA viral infection is implicated.

The combination product of the invention is an antiviral agent.

The pharmaceutical composition of the invention is an antiviral agent.

In some embodiments, the combination product, pharmaceutical compositions, and methods disclosed herein are used in the prevention or treatment of a viral infection. Exemplary viral infection includes but are not limited to SARS-CoV-2 viral infection.

The combination product of the invention is also useful in treating viral infections caused by RNA viruses. For example, viral infections treatable according to the methods of the invention caused by Measles, Mumps, Respiratory Syncytial Virus, SARS-CoV-2, Metapneumovirus, Poliovirus, Chikungunya virus, Hepatitis A virus, Hepatitis C virus, Hepatitis E virus, West Nile virus, Zika virus, Dengue virus, Lassa Fever virus, Junin South American hemorrhagic fever virus, Marburg virus, Ebola virus, Norovirus (human caliciviruses) infections including the Norwalk agent, Rift Valley Fever virus, Nipah virus, Hendra virus, HIV-1, HTLV-1, HTLV-2, rotavirus, seasonal and pandemic coronaviruses, Picobirnaviruses, Toroviruses, and Astroviruses.

In some embodiments, the viral infection is SARS-CoV-2 viral infection.

In some embodiments, the viral infection is Measles viral infection.

In some embodiments, the viral infection is Mumps viral infection.

In some embodiments, the viral infection is Respiratory Syncytial Virus infection.

In some embodiments, the viral infection is Metapneumovirus infection.

In some embodiments, the viral infection is Poliovirus infection.

In some embodiments, the viral infection is Chikungunya virus infection.

In some embodiments, the viral infection is Hepatitis A virus infection.

In some embodiments, the viral infection is Hepatitis C virus infection.

In some embodiments, the viral infection is Hepatitis E virus infection.

In some embodiments, the viral infection is West Nile virus infection.

In some embodiments, the viral infection is Zika virus infection.

In some embodiments, the viral infection is Dengue virus infection.

In some embodiments, the viral infection is Lassa Fever virus infection.

In some embodiments, the viral infection is Junin South American hemorrhagic fever virus infection.

In some embodiments, the viral infection is Marburg virus infection.

In some embodiments, the viral infection is Ebola virus infection.

In some embodiments, the viral infection is Norovirus (human caliciviruses) infections including the Norwalk agent virus infection.

In some embodiments, the viral infection is Rift Valley Fever virus infection.

In some embodiments, the viral infection is Nipah virus infection.

In some embodiments, the viral infection is Hendra virus infection.

In some embodiments, the viral infection is HIV-1 infection.

In some embodiments, the viral infection is HTLV-1 infection.

In some embodiments, the viral infection is HTLV-2 infection.

In some embodiments, the viral infection is rotavirus infection.

In some embodiments, the viral infection is seasonal coronaviruses infection.

In some embodiments, the viral infection is pandemic coronaviruses infection.

In some embodiments, the viral infection is Picobirnaviruses infection.

In some embodiments, the viral infection is Toroviruses infection.

In some embodiments, the viral infection is Astroviruses infection.

In some aspects, the present disclosure provides a method of treating or preventing a SARS-CoV-2 viral infection in a subject in need thereof, comprising administering to the subject a therapeutically effective amount of a combination product of the present disclosure or a pharmaceutical composition of the present disclosure.

In some aspects, the present disclosure provides a method of treating or preventing a Measles viral infection in a subject in need thereof, comprising administering to the subject a therapeutically effective amount of a combination product of the present disclosure or a pharmaceutical composition of the present disclosure.

In some aspects, the present disclosure provides a method of treating or preventing a Mumps viral infection in a subject in need thereof, comprising administering to the subject a therapeutically effective amount of a combination product of the present disclosure or a pharmaceutical composition of the present disclosure.

In some aspects, the present disclosure provides a method of treating or preventing a Respiratory Syncytial Virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Metapneumovirus infection in a subject in need thereof, comprising administering to the subject a therapeutically effective amount of a combination product of the present disclosure or a pharmaceutical composition of the present disclosure.

In some aspects, the present disclosure provides a method of treating or preventing a Poliovirus infection in a subject in need thereof, comprising administering to the subject a therapeutically effective amount of a combination product of the present disclosure or a pharmaceutical composition of the present disclosure.

In some aspects, the present disclosure provides a method of treating or preventing a Chikungunya virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Hepatitis A virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Hepatitis C virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Hepatitis E virus infection in a subject in need thereof, comprising administering to the subject a therapeutically effective amount of a combination product of the present disclosure or a pharmaceutical composition of the present disclosure.

In some aspects, the present disclosure provides a method of treating or preventing a West Nile virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Zika virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Dengue virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Lassa Fever virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Junin South American hemorrhagic fever virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Marburg virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Ebola virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Norovirus (human caliciviruses) infections including the Norwalk agent virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Rift Valley Fever virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Nipah virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Hendra virus infection in a subject in need thereof, comprising administering to the subject a therapeutically effective amount of a combination product of the present disclosure or a pharmaceutical composition of the present disclosure.

In some aspects, the present disclosure provides a method of treating or preventing a HIV-1 infection in a subject in need thereof, comprising administering to the subject a therapeutically effective amount of a combination product of the present disclosure or a pharmaceutical composition of the present disclosure.

In some aspects, the present disclosure provides a method of treating or preventing a HTLV-1 infection in a subject in need thereof, comprising administering to the subject a therapeutically effective amount of a combination product of the present disclosure or a pharmaceutical composition of the present disclosure.

In some aspects, the present disclosure provides a method of treating or preventing a HTLV-2 infection in a subject in need thereof, comprising administering to the subject a therapeutically effective amount of a combination product of the present disclosure or a pharmaceutical composition of the present disclosure.

In some aspects, the present disclosure provides a method of treating or preventing a rotavirus infection in a subject in need thereof, comprising administering to the subject a therapeutically effective amount of a combination product of the present disclosure or a pharmaceutical composition of the present disclosure.

In some aspects, the present disclosure provides a method of treating or preventing a seasonal coronaviruses infection in a subject in need thereof, comprising administering to the subject a therapeutically effective amount of a combination product of the present disclosure or a pharmaceutical composition of the present disclosure.

In some aspects, the present disclosure provides a method of treating or preventing a pandemic coronaviruses infection in a subject in need thereof, comprising administering to the subject a therapeutically effective amount of a combination product of the present disclosure or a pharmaceutical composition of the present disclosure.

In some aspects, the present disclosure provides a method of treating or preventing a Picobirnaviruses infection in a subject in need thereof, comprising administering to the subject a therapeutically effective amount of a combination product of the present disclosure or a pharmaceutical composition of the present disclosure.

In some aspects, the present disclosure provides a method of treating or preventing a Toroviruses infection in a subject in need thereof, comprising administering to the subject a therapeutically effective amount of a combination product of the present disclosure or a pharmaceutical composition of the present disclosure.

In some aspects, the present disclosure provides a method of treating or preventing a Astroviruses infection in a subject in need thereof, comprising administering to the subject a therapeutically effective amount of a combination product of the present disclosure or a pharmaceutical composition of the present disclosure.

In some aspects, the present disclosure provides a method of treating a SARS-CoV-2 viral infection in a subject in need thereof, comprising administering to the subject a therapeutically effective amount of a combination product of the present disclosure or a pharmaceutical composition of the present disclosure.

In some aspects, the present disclosure provides a method of treating a Measles viral infection in a subject in need thereof, comprising administering to the subject a therapeutically effective amount of a combination product of the present disclosure or a pharmaceutical composition of the present disclosure.

In some aspects, the present disclosure provides a method of treating a Mumps viral infection in a subject in need thereof, comprising administering to the subject a therapeutically effective amount of a combination product of the present disclosure or a pharmaceutical composition of the present disclosure.

In some aspects, the present disclosure provides a method of treating a Respiratory Syncytial Virus infection in a subject in need thereof, comprising administering to the subject a therapeutically effective amount of a combination product of the present disclosure or a pharmaceutical composition of the present disclosure.

In some aspects, the present disclosure provides a method of treating a Metapneumovirus infection in a subject in need thereof, comprising administering to the subject a therapeutically effective amount of a combination product of the present disclosure or a pharmaceutical composition of the present disclosure.

In some aspects, the present disclosure provides a method of treating a Poliovirus infection in a subject in need thereof, comprising administering to the subject a therapeutically effective amount of a combination product of the present disclosure or a pharmaceutical composition of the present disclosure.

In some aspects, the present disclosure provides a method of treating a Chikungunya virus infection in a subject in need thereof, comprising administering to the subject a therapeutically effective amount of a combination product of the present disclosure or a pharmaceutical composition of the present disclosure.

In some aspects, the present disclosure provides a method of treating a Hepatitis A virus infection in a subject in need thereof, comprising administering to the subject a therapeutically effective amount of a combination product of the present disclosure or a pharmaceutical composition of the present disclosure.

In some aspects, the present disclosure provides a method of treating a Hepatitis C virus infection in a subject in need thereof, comprising administering to the subject a therapeutically effective amount of a combination product of the present disclosure or a pharmaceutical composition of the present disclosure.

In some aspects, the present disclosure provides a method of treating a Hepatitis E virus infection in a subject in need thereof, comprising administering to the subject a therapeutically effective amount of a combination product of the present disclosure or a pharmaceutical composition of the present disclosure.

In some aspects, the present disclosure provides a method of treating a West Nile virus infection in a subject in need thereof, comprising administering to the subject a therapeutically effective amount of a combination product of the present disclosure or a pharmaceutical composition of the present disclosure.

In some aspects, the present disclosure provides a method of treating a Zika virus infection in a subject in need thereof, comprising administering to the subject a therapeutically effective amount of a combination product of the present disclosure or a pharmaceutical composition of the present disclosure.

In some aspects, the present disclosure provides a method of treating a Dengue virus infection in a subject in need thereof, comprising administering to the subject a therapeutically effective amount of a combination product of the present disclosure or a pharmaceutical composition of the present disclosure.

In some aspects, the present disclosure provides a method of treating a Lassa Fever virus infection in a subject in need thereof, comprising administering to the subject a therapeutically effective amount of a combination product of the present disclosure or a pharmaceutical composition of the present disclosure.

In some aspects, the present disclosure provides a method of treating a Junin South American hemorrhagic fever virus infection in a subject in need thereof, comprising administering to the subject a therapeutically effective amount of a combination product of the present disclosure or a pharmaceutical composition of the present disclosure.

In some aspects, the present disclosure provides a method of treating a Marburg virus infection in a subject in need thereof, comprising administering to the subject a therapeutically effective amount of a combination product of the present disclosure or a pharmaceutical composition of the present disclosure.

In some aspects, the present disclosure provides a method of treating a Ebola virus infection in a subject in need thereof, comprising administering to the subject a therapeutically effective amount of a combination product of the present disclosure or a pharmaceutical composition of the present disclosure.

In some aspects, the present disclosure provides a method of treating a Norovirus (human caliciviruses) infections including the Norwalk agent virus infection in a subject in need thereof, comprising administering to the subject a therapeutically effective amount of a combination product of the present disclosure or a pharmaceutical composition of the present disclosure.

In some aspects, the present disclosure provides a method of treating a Rift Valley Fever virus infection in a subject in need thereof, comprising administering to the subject a therapeutically effective amount of a combination product of the present disclosure or a pharmaceutical composition of the present disclosure.

In some aspects, the present disclosure provides a method of treating a Nipah virus infection in a subject in need thereof, comprising administering to the subject a therapeutically effective amount of a combination product of the present disclosure or a pharmaceutical composition of the present disclosure.

In some aspects, the present disclosure provides a method of treating a Hendra virus infection in a subject in need thereof, comprising administering to the subject a therapeutically effective amount of a combination product of the present disclosure or a pharmaceutical composition of the present disclosure.

In some aspects, the present disclosure provides a method of treating a HIV-1 infection in a subject in need thereof, comprising administering to the subject a therapeutically effective amount of a combination product of the present disclosure or a pharmaceutical composition of the present disclosure.

In some aspects, the present disclosure provides a method of treating a HTLV-1 infection in a subject in need thereof, comprising administering to the subject a therapeutically effective amount of a combination product of the present disclosure or a pharmaceutical composition of the present disclosure.

In some aspects, the present disclosure provides a method of treating a HTLV-2 infection in a subject in need thereof, comprising administering to the subject a therapeutically effective amount of a combination product of the present disclosure or a pharmaceutical composition of the present disclosure.

In some aspects, the present disclosure provides a method of treating a rotavirus infection in a subject in need thereof, comprising administering to the subject a therapeutically effective amount of a combination product of the present disclosure or a pharmaceutical composition of the present disclosure.

In some aspects, the present disclosure provides a method of treating a seasonal coronaviruses infection in a subject in need thereof, comprising administering to the subject a therapeutically effective amount of a combination product of the present disclosure or a pharmaceutical composition of the present disclosure.

In some aspects, the present disclosure provides a method of treating a pandemic coronaviruses infection in a subject in need thereof, comprising administering to the subject a therapeutically effective amount of a combination product of the present disclosure or a pharmaceutical composition of the present disclosure.

In some aspects, the present disclosure provides a method of treating a Picobirnaviruses infection in a subject in need thereof, comprising administering to the subject a therapeutically effective amount of a combination product of the present disclosure or a pharmaceutical composition of the present disclosure.

In some aspects, the present disclosure provides a method of treating a Toroviruses infection in a subject in need thereof, comprising administering to the subject a therapeutically effective amount of a combination product of the present disclosure or a pharmaceutical composition of the present disclosure.

In some aspects, the present disclosure provides a method of treating a Astroviruses infection in a subject in need thereof, comprising administering to the subject a therapeutically effective amount of a combination product of the present disclosure or a pharmaceutical composition of the present disclosure.

In some aspects, the present disclosure provides a combination product of the present disclosure for use in inhibiting of virus RNA replication (e.g., in vitro or in vivo).

In some aspects, the present disclosure provides a combination product of the present disclosure for use in treating or preventing a disease or disorder disclosed herein.

In some aspects, the present disclosure provides a combination product of the present disclosure for use in treating or preventing a viral infection disclosed herein.

In some aspects, the present disclosure provides a combination product of the present disclosure for use in treating a disease or disorder disclosed herein.

In some aspects, the present disclosure provides a combination product of the present disclosure for use in treating a viral infection disclosed herein.

In some aspects, the present disclosure provides a combination product of the present disclosure for use in treating or preventing a SARS-CoV-2 viral infection in a subject in need thereof.

In some aspects, the present disclosure provides a combination product of the present disclosure for use in treating or preventing a Measles viral infection in a subject in need thereof.

In some aspects, the present disclosure provides a combination product of the present disclosure for use in treating or preventing a Mumps viral infection in a subject in need thereof.

In some aspects, the present disclosure provides a combination product of the present disclosure for use in treating or preventing a Respiratory Syncytial Virus infection in a subject in need thereof.

In some aspects, the present disclosure provides a combination product of the present disclosure for use in treating or preventing a Metapneumovirus infection in a subject in need thereof.

In some aspects, the present disclosure provides a combination product of the present disclosure for use in treating or preventing a Poliovirus infection in a subject in need thereof.

In some aspects, the present disclosure provides a combination product of the present disclosure for use in treating or preventing a Chikungunya virus infection in a subject in need thereof.

In some aspects, the present disclosure provides a combination product of the present disclosure for use in treating or preventing a Hepatitis A virus infection in a subject in need thereof.

In some aspects, the present disclosure provides a combination product of the present disclosure for use in treating or preventing a Hepatitis C virus infection in a subject in need thereof.

In some aspects, the present disclosure provides a combination product of the present disclosure for use in treating or preventing a Hepatitis E virus infection in a subject in need thereof.

In some aspects, the present disclosure provides a combination product of the present disclosure for use in treating or preventing a West Nile virus infection in a subject in need thereof.

In some aspects, the present disclosure provides a combination product of the present disclosure for use in treating or preventing a Zika virus infection in a subject in need thereof.

In some aspects, the present disclosure provides a combination product of the present disclosure for use in treating or preventing a Dengue virus infection in a subject in need thereof.

In some aspects, the present disclosure provides a combination product of the present disclosure for use in treating or preventing a Lassa Fever virus infection in a subject in need thereof.

In some aspects, the present disclosure provides a combination product of the present disclosure for use in treating or preventing a Junin South American hemorrhagic fever virus infection in a subject in need thereof.

In some aspects, the present disclosure provides a combination product of the present disclosure for use in treating or preventing a Marburg virus infection in a subject in need thereof.

In some aspects, the present disclosure provides a combination product of the present disclosure for use in treating or preventing a Ebola virus infection in a subject in need thereof.

In some aspects, the present disclosure provides a combination product of the present disclosure for use in treating or preventing a Norovirus (human caliciviruses) infections including the Norwalk agent virus infection in a subject in need thereof.

In some aspects, the present disclosure provides a combination product of the present disclosure for use in treating or preventing a Rift Valley Fever virus infection in a subject in need thereof.

In some aspects, the present disclosure provides a combination product of the present disclosure for use in treating or preventing a Nipah virus infection in a subject in need thereof.

In some aspects, the present disclosure provides a combination product of the present disclosure for use in treating or preventing a Hendra virus infection in a subject in need thereof.

In some aspects, the present disclosure provides a combination product of the present disclosure for use in treating or preventing a HIV-1 infection in a subject in need thereof.

In some aspects, the present disclosure provides a combination product of the present disclosure for use in treating or preventing a HTLV-1 infection in a subject in need thereof.

In some aspects, the present disclosure provides a combination product of the present disclosure for use in treating or preventing a HTLV-2 infection in a subject in need thereof.

In some aspects, the present disclosure provides a combination product of the present disclosure for use in treating or preventing a rotavirus infection in a subject in need thereof.

In some aspects, the present disclosure provides a combination product of the present disclosure for use in treating or preventing a seasonal coronaviruses infection in a subject in need thereof.

In some aspects, the present disclosure provides a combination product of the present disclosure for use in treating or preventing a pandemic coronaviruses infection in a subject in need thereof.

In some aspects, the present disclosure provides a combination product of the present disclosure for use in treating or preventing a Picobirnaviruses infection in a subject in need thereof.

In some aspects, the present disclosure provides a combination product of the present disclosure for use in treating or preventing a Toroviruses infection in a subject in need thereof.

In some aspects, the present disclosure provides a combination product of the present disclosure for use in treating or preventing a Astroviruses infection in a subject in need thereof.

In some aspects, the present disclosure provides a combination product of the present disclosure for use in treating a SARS-CoV-2 viral infection in a subject in need thereof.

In some aspects, the present disclosure provides a combination product of the present disclosure for use in treating a Measles viral infection in a subject in need thereof.

In some aspects, the present disclosure provides a combination product of the present disclosure for use in treating a Mumps viral infection in a subject in need thereof.

In some aspects, the present disclosure provides a combination product of the present disclosure for use in treating a Respiratory Syncytial Virus infection in a subject in need thereof.

In some aspects, the present disclosure provides a combination product of the present disclosure for use in treating a Metapneumovirus infection in a subject in need thereof.

In some aspects, the present disclosure provides a combination product of the present disclosure for use in treating a Poliovirus infection in a subject in need thereof.

In some aspects, the present disclosure provides a combination product of the present disclosure for use in treating a Chikungunya virus infection in a subject in need thereof.

In some aspects, the present disclosure provides a combination product of the present disclosure for use in treating a Hepatitis A virus infection in a subject in need thereof.

In some aspects, the present disclosure provides a combination product of the present disclosure for use in treating a Hepatitis C virus infection in a subject in need thereof.

In some aspects, the present disclosure provides a combination product of the present disclosure for use in treating a Hepatitis E virus infection in a subject in need thereof.

In some aspects, the present disclosure provides a combination product of the present disclosure for use in treating a West Nile virus infection in a subject in need thereof.

In some aspects, the present disclosure provides a combination product of the present disclosure for use in treating a Zika virus infection in a subject in need thereof.

In some aspects, the present disclosure provides a combination product of the present disclosure for use in treating a Dengue virus infection in a subject in need thereof.

In some aspects, the present disclosure provides a combination product of the present disclosure for use in treating a Lassa Fever virus infection in a subject in need thereof.

In some aspects, the present disclosure provides a combination product of the present disclosure for use in treating a Junin South American hemorrhagic fever virus infection in a subject in need thereof.

In some aspects, the present disclosure provides a combination product of the present disclosure for use in treating a Marburg virus infection in a subject in need thereof.

In some aspects, the present disclosure provides a combination product of the present disclosure for use in treating a Ebola virus infection in a subject in need thereof.

In some aspects, the present disclosure provides a combination product of the present disclosure for use in treating a Norovirus (human caliciviruses) infections including the Norwalk agent virus infection in a subject in need thereof.

In some aspects, the present disclosure provides a combination product of the present disclosure for use in treating a Rift Valley Fever virus infection in a subject in need thereof.

In some aspects, the present disclosure provides a combination product of the present disclosure for use in treating a Nipah virus infection in a subject in need thereof.

In some aspects, the present disclosure provides a combination product of the present disclosure for use in treating a Hendra virus infection in a subject in need thereof.

In some aspects, the present disclosure provides a combination product of the present disclosure for use in treating a HIV-1 infection in a subject in need thereof.

In some aspects, the present disclosure provides a combination product of the present disclosure for use in treating a HTLV-1 infection in a subject in need thereof.

In some aspects, the present disclosure provides a combination product of the present disclosure for use in treating a HTLV-2 infection in a subject in need thereof.

In some aspects, the present disclosure provides a combination product of the present disclosure for use in treating a rotavirus infection in a subject in need thereof.

In some aspects, the present disclosure provides a combination product of the present disclosure for use in treating a seasonal coronaviruses infection in a subject in need thereof.

In some aspects, the present disclosure provides a combination product of the present disclosure for use in treating a pandemic coronaviruses infection in a subject in need thereof.

In some aspects, the present disclosure provides a combination product of the present disclosure for use in treating a Picobirnaviruses infection in a subject in need thereof.

In some aspects, the present disclosure provides a combination product of the present disclosure for use in treating a Toroviruses infection in a subject in need thereof.

In some aspects, the present disclosure provides a combination product of the present disclosure for use in treating a Astroviruses infection in a subject in need thereof.

In some aspects, the present disclosure provides use of a combination product of the present disclosure in the manufacture of a medicament for inhibiting viral RNA replication (e.g., in vitro or in vivo).

In some aspects, the present disclosure provides use of a combination product of the present disclosure or a pharmaceutical composition thereof in the manufacture of a medicament for treating or preventing a disease or disorder disclosed herein.

In some aspects, the present disclosure provides use of a pharmaceutical composition of the present disclosure in the manufacture of a medicament for treating or preventing a disease or disorder disclosed herein.

In some aspects, the present disclosure provides use of a combination product of the present disclosure in the manufacture of a medicament for treating or preventing a viral infection disclosed herein.

In some aspects, the present disclosure provides use of a combination product of the present disclosure or a pharmaceutical composition thereof in the manufacture of a medicament for treating a disease or disorder disclosed herein.

In some aspects, the present disclosure provides use of a combination product of the present disclosure in the manufacture of a medicament for treating a viral infection disclosed herein.

In some aspects, the present disclosure provides use of a pharmaceutical composition of the present disclosure in the manufacture of a medicament for treating a viral infection disclosed herein.

In some aspects, the present disclosure provides use of a combination product of the present disclosure in the manufacture of a medicament for treating or preventing a SARS-CoV-2 viral infection in a subject in need thereof.

In some aspects, the present disclosure provides use of a combination product of the present disclosure in the manufacture of a medicament for treating or preventing a Measles viral infection in a subject in need thereof.

In some aspects, the present disclosure provides use of a combination product of the present disclosure in the manufacture of a medicament for treating or preventing a Mumps viral infection in a subject in need thereof.

In some aspects, the present disclosure provides use of a combination product of the present disclosure in the manufacture of a medicament for treating or preventing a Respiratory Syncytial Virus infection in a subject in need thereof.

In some aspects, the present disclosure provides use of a combination product of the present disclosure in the manufacture of a medicament for treating or preventing a Metapneumovirus infection in a subject in need thereof.

In some aspects, the present disclosure provides use of a combination product of the present disclosure in the manufacture of a medicament for treating or preventing a Poliovirus infection in a subject in need thereof.

In some aspects, the present disclosure provides use of a combination product of the present disclosure in the manufacture of a medicament for treating or preventing a Chikungunya virus infection in a subject in need thereof.

In some aspects, the present disclosure provides use of a combination product of the present disclosure in the manufacture of a medicament for treating or preventing a Hepatitis A virus infection in a subject in need thereof.

In some aspects, the present disclosure provides use of a combination product of the present disclosure in the manufacture of a medicament for treating or preventing a Hepatitis C virus infection in a subject in need thereof.

In some aspects, the present disclosure provides use of a combination product of the present disclosure in the manufacture of a medicament for treating or preventing a Hepatitis E virus infection in a subject in need thereof.

In some aspects, the present disclosure provides use of a combination product of the present disclosure in the manufacture of a medicament for treating or preventing a West Nile virus infection in a subject in need thereof.

In some aspects, the present disclosure provides use of a combination product of the present disclosure in the manufacture of a medicament for treating or preventing a Zika virus infection in a subject in need thereof.

In some aspects, the present disclosure provides use of a combination product of the present disclosure in the manufacture of a medicament for treating or preventing a Dengue virus infection in a subject in need thereof.

In some aspects, the present disclosure provides use of a combination product of the present disclosure in the manufacture of a medicament for treating or preventing a Lassa Fever virus infection in a subject in need thereof.

In some aspects, the present disclosure provides use of a combination product of the present disclosure in the manufacture of a medicament for treating or preventing a Junin South American hemorrhagic fever virus infection in a subject in need thereof.

In some aspects, the present disclosure provides use of a combination product of the present disclosure in the manufacture of a medicament for treating or preventing a Marburg virus infection in a subject in need thereof.

In some aspects, the present disclosure provides use of a combination product of the present disclosure in the manufacture of a medicament for treating or preventing a Ebola virus infection in a subject in need thereof.

In some aspects, the present disclosure provides use of a combination product of the present disclosure in the manufacture of a medicament for treating or preventing a Norovirus (human caliciviruses) infections including the Norwalk agent virus infection in a subject in need thereof.

In some aspects, the present disclosure provides use of a combination product of the present disclosure in the manufacture of a medicament for treating or preventing a Rift Valley Fever virus infection in a subject in need thereof.

In some aspects, the present disclosure provides use of a combination product of the present disclosure in the manufacture of a medicament for treating or preventing a Nipah virus infection in a subject in need thereof.

In some aspects, the present disclosure provides use of a combination product of the present disclosure in the manufacture of a medicament for treating or preventing a Hendra virus infection in a subject in need thereof.

In some aspects, the present disclosure provides use of a combination product of the present disclosure in the manufacture of a medicament for treating or preventing a HIV-1 infection in a subject in need thereof.

In some aspects, the present disclosure provides use of a combination product of the present disclosure in the manufacture of a medicament for treating or preventing a HTLV-1 infection in a subject in need thereof.

In some aspects, the present disclosure provides use of a combination product of the present disclosure in the manufacture of a medicament for treating or preventing a HTLV-2 infection in a subject in need thereof.

In some aspects, the present disclosure provides use of a combination product of the present disclosure in the manufacture of a medicament for treating or preventing a rotavirus infection in a subject in need thereof.

In some aspects, the present disclosure provides use of a combination product of the present disclosure in the manufacture of a medicament for treating or preventing a seasonal coronaviruses infection in a subject in need thereof.

In some aspects, the present disclosure provides use of a combination product of the present disclosure in the manufacture of a medicament for treating or preventing a pandemic coronaviruses infection in a subject in need thereof.

In some aspects, the present disclosure provides use of a combination product of the present disclosure in the manufacture of a medicament for treating or preventing a Picobirnaviruses infection in a subject in need thereof.

In some aspects, the present disclosure provides use of a combination product of the present disclosure in the manufacture of a medicament for treating or preventing a Toroviruses infection in a subject in need thereof.

In some aspects, the present disclosure provides use of a combination product of the present disclosure in the manufacture of a medicament for treating or preventing a Astroviruses infection in a subject in need thereof.

The present disclosure provides combination products and pharmaceutical compositions that function as inhibitors of viral RNA replication (e.g., in vitro or in vivo).

The present disclosure therefore provides a method of inhibiting of viral RNA replication in vitro or in vivo, said method comprising contacting a cell with a therapeutically effective amount of a compound, or a pharmaceutically acceptable salt thereof, as defined herein.

In some embodiments, the inhibitor of viral RNA replication is a combination product of the present disclosure.

In some embodiments, the inhibitor of viral RNA replication is a pharmaceutical composition of the present disclosure.

Industry-accepted assays/disease models can determine effectiveness of combination products of the disclosure according to standard practices of elucidating the same as described in the art and are found in the current general knowledge.

The present disclosure also provides a method of treating a viral infection in a subject in need of such treatment, said method comprising administering to said subject a therapeutically effective amount of a combination product or a pharmaceutical composition as defined herein.

Another aspect of the invention relates to lowering the dose of (i) favipiravir needed to increase inhibition of SARS-CoV-2 virus replication.

Another aspect of the invention relates to lowering the dose of (ii) IMPDH inhibitor needed to increase inhibition of SARS-CoV-2 virus replication.

Another aspect of the invention relates to lowering the dose of (ii) mycophenolate mofetil needed to increase inhibition of SARS-CoV-2 virus replication.

Another aspect of the invention relates to lowering the dose of (ii) viramidine needed to increase inhibition of SARS-CoV-2 virus replication.

Another aspect of the invention relates to use of combination of (i) favipiravir and (ii) IMPDH inhibitor, for treating seasonal, pandemic and drug-resistant influenza virus disease.

Another aspect of the invention relates to use of combination of (i) favipiravir and (ii) mycophenolate mofetil, for treating seasonal, pandemic and drug-resistant influenza virus disease.

Another aspect of the invention relates to use of combination of (i) favipiravir and (ii) viramidine, for treating seasonal, pandemic and drug-resistant influenza virus disease.

Another aspect of the invention relates to use of combination of (i) favipiravir and (ii) inhibitors of Inosine monophosphate dehydrogenase for treating diseases caused by RNA virus infection including Measles, Mumps, Respiratory Syncytial Virus, Metapneumovirus, Poliovirus, Chikungunya virus, Hepatitis A virus, Hepatitis C virus, Hepatitis E virus, West Nile virus, Zika virus, Dengue virus, Lassa Fever virus, Junin South American hemorrhagic fever virus, Marburg virus, Ebola virus, Norovirus (human caliciviruses) infections including the Norwalk agent, Rift Valley Fever virus, Nipah virus, Hendra virus, HIV-1, HTLV-1, HTLV-2, rotavirus, seasonal and pandemic coronaviruses, Picobirnaviruses, Toroviruses, and Astroviruses.

Another aspect of the invention relates to use of combination of (i) favipiravir and (ii) mycophenolate mofetil for treating diseases caused by RNA virus infection including Measles, Mumps, Respiratory Syncytial Virus, Metapneumovirus, Poliovirus, Chikungunya virus, Hepatitis A virus, Hepatitis C virus, Hepatitis E virus, West Nile virus, Zika virus, Dengue virus, Lassa Fever virus, Junin South American hemorrhagic fever virus, Marburg virus, Ebola virus, Norovirus (human caliciviruses) infections including the Norwalk agent, Rift Valley Fever virus, Nipah virus, Hendra virus, HIV-1, HTLV-1, HTLV-2, rotavirus, seasonal and pandemic coronaviruses, Picobirnaviruses, Toroviruses, and Astroviruses.

Another aspect of the invention relates to use of combination of (i) favipiravir and (ii) viramidine for treating diseases caused by RNA virus infection including Measles,

Mumps, Respiratory Syncytial Virus, Metapneumovirus, Poliovirus, Chikungunya virus, Hepatitis A virus, Hepatitis C virus, Hepatitis E virus, West Nile virus, Zika virus, Dengue virus, Lassa Fever virus, Junin South American hemorrhagic fever virus, Marburg virus, Ebola virus, Norovirus (human caliciviruses) infections including the Norwalk agent, Rift Valley Fever virus, Nipah virus, Hendra virus, HIV-1, HTLV-1, HTLV-2, rotavirus, seasonal and pandemic coronaviruses, Picobimaviruses, Toroviruses, and Astroviruses.

In some embodiments, the subject is a mammal.

In some embodiments, the subject is a human.

### Routes of Administration

The combination products of the disclosure or pharmaceutical compositions comprising these combination products may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.): intranasal (e.g., by nasal spray); ocular (e.g., by eye drops): pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Examples of Combination Products

In the Table 3 presented certain non-limiting examples of the combination products.

TABLE 3
Selected examples of the combination products

| | Inhibitor of viral | | Molar | Dose, |
|---|---|---|---|---|
| # | RNA replication | IMPDH inhibitor | ratio | mg/kg |
| 1 | favipiravir | mycophenolate mofetil | 1:1 | 0.3 |
| 2 | favipiravir | mycophenolate mofetil | 1:2 | 0.6 |
| 3 | favipiravir | mycophenolate mofetil | 1:5 | 1.5 |
| 4 | favipiravir | mycophenolate mofetil | 1:10 | 3 |
| 5 | favipiravir | mycophenolate mofetil | 1:50 | 15 |
| 6 | favipiravir | mycophenolate mofetil | 1:100 | 30 |
| 7 | favipiravir | mycophenolate mofetil | 2:1 | 0.6 |
| 8 | favipiravir | mycophenolate mofetil | 5:1 | 1.5 |
| 9 | favipiravir | mycophenolate mofetil | 10:1 | 3 |
| 10 | favipiravir | mycophenolate mofetil | 50:1 | 15 |
| 11 | favipiravir | mycophenolate mofetil | 100:1 | 30 |
| 12 | favipiravir | ribavirin | 5:1 | 15 |
| 13 | favipiravir | mycophenolic acid (MPA) | 25:1 | 15 |
| 14 | favipiravir | merimepodib (VX-497) | 10:1 | 15 |
| 15 | favipiravir | viramidine | 1:1 | 0.3 |
| 16 | favipiravir | viramidine | 1:2 | 0.6 |
| 17 | favipiravir | viramidine | 1:5 | 1.5 |
| 18 | favipiravir | viramidine | 1:10 | 3 |
| 19 | favipiravir | viramidine | 1:50 | 15 |
| 20 | favipiravir | viramidine | 1:100 | 30 |
| 21 | favipiravir | viramidine | 2:1 | 0.6 |
| 22 | favipiravir | viramidine | 5:1 | 1.5 |
| 23 | favipiravir | viramidine | 10:1 | 3 |
| 24 | favipiravir | viramidine | 50:1 | 15 |
| 25 | favipiravir | viramidine | 100:1 | 30 |
| 26 | favipiravir | mizoribine | 10:1 | 15 |
| 27 | favipiravir | 5-ethyl-1-beta-d-ribo- | 10:1 | 15 |
| | | furanosylimidazole-4- | | |
| | | carboamide (EICAR) | | |
| 28 | favipiravir | 6-chloropurin riboside | 10:1 | 15 |
| 29 | favipiravir | 3-deazaguanosine | 10:1 | 15 |
| 30 | favipiravir | 2-vinylinosines | 10:1 | 15 |
| 31 | favipiravir | thiazole-4-carboxamide | 10:1 | 15 |
| | | dinucleotide | | |
| 32 | favipiravir | tiazofurin | 10:1 | 15 |
| 33 | favipiravir | 2-(fluorovinyl)inosine | 10:1 | 15 |
| | | monophosphate | | |
| 34 | favipiravir | selenazofurin | 10:1 | 15 |
| 35 | favipiravir | benzamidine riboside | 10:1 | 15 |
| 36 | favipiravir | VX-148 | 10:1 | 15 |
| 37 | favipiravir-NTP | mycophenolate mofetil | 1:1 | 0.3 |
| 38 | favipiravir-NTP | mycophenolate mofetil | 1:2 | 0.6 |
| 39 | favipiravir-NTP | mycophenolate mofetil | 1:5 | 1.5 |
| 40 | favipiravir-NTP | mycophenolate mofetil | 1:10 | 3 |
| 41 | favipiravir-NTP | mycophenolate mofetil | 1:50 | 15 |
| 42 | favipiravir-NTP | mycophenolate mofetil | 1:100 | 30 |
| 43 | favipiravir-NTP | mycophenolate mofetil | 2:1 | 0.6 |
| 44 | favipiravir-NTP | mycophenolate mofetil | 5:1 | 1.5 |
| 45 | favipiravir-NTP | mycophenolate mofetil | 10:1 | 3 |
| 46 | favipiravir-NTP | mycophenolate mofetil | 50:1 | 15 |
| 47 | favipiravir-NTP | mycophenolate mofetil | 100:1 | 30 |
| 48 | favipiravir-NTP | ribavirin | 5:1 | 15 |
| 49 | favipiravir-NTP | mycophenolic acid (MPA) | 25:1 | 15 |
| 50 | favipiravir-NTP | merimepodib (VX-497) | 10:1 | 15 |
| 51 | favipiravir-NTP | viramidine | 2:1 | 15 |
| 52 | favipiravir-NTP | mizoribine | 10:1 | 15 |
| 53 | favipiravir-NTP | 5-ethyl-1-beta-d-ribo- | 2:1 | 15 |
| | | furanosylimidazole-4- | | |
| | | carboamide (EICAR) | | |
| 54 | favipiravir-NTP | 6-chloropurin riboside | 10:1 | 15 |
| 55 | favipiravir-NTP | 3-deazaguanosine | 10:1 | 15 |
| 56 | favipiravir-NTP | 2-vinylinosines | 10:1 | 15 |
| 57 | favipiravir-NTP | thiazole-4-carboxamide | 10:1 | 15 |
| | | dinucleotide | | |
| 58 | favipiravir-NTP | tiazofurin | 10:1 | 15 |
| 59 | favipiravir-NTP | 2-(fluorovinyl)inosine | 10:1 | 15 |
| | | monophosphate | | |
| 60 | favipiravir-NTP | selenazofurin | 10:1 | 15 |
| 61 | favipiravir-NTP | benzamidine riboside | 10:1 | 15 |
| 62 | favipiravir-NTP | VX-148 | 10:1 | 15 |

### Examples of Pharmaceutical Compositions

Certain non-limiting examples of the pharmaceutical compositions of the combination products described here

The pharmaceutical composition according to disclosure comprises combination product and auxiliary compounds.

The examples of combination products according to invention presented in the Table 3.

According to an embodiment of the present invention, the pharmaceutical composition may be in the form of tablets, capsules, tablets filled in capsule, minitablets filled in capsule, sachets containing powder or granules, pellets, and the like. The pharmaceutical composition is meant for once daily or twice daily administration.

The preparation of film coated tablets having some of the auxiliary ingredients as listed below which can either include or exclude when prepared using conventional techniques:
Sodium lauryl sulfate
Microcrystalline cellulose
Lactose monohydrate
Lactose 200 mesh
Croscarmellose sodium
Hydroxypropyl methyl cellulose (Hypromellose)
Sodium carboxymethylcellulose
Talc
Magnesium stearate
Lactose DC
Maize starch
Pregelatinized starch
Colloidal silicon dioxide
Polyethylene glycol 6000 (macrogol)
Basic calcium phosphate
Menitol
Ethyl cellulose
Hydroxypropyl methylcellulose k100
Light magnesium oxide
Magnesium hydroxide
Calcium CMC
Hydrogenated castor oil
Sodium starch glycolate
Sodium benzoate
Sodium methyl paraben sodium propyl paraben
PVP k30
PVP k90
FILM COAT
Titanium dioxide.

### Biological Assays

### Example A. Inhibition Test for Flu Virus (and Other Respiratory Viruses)

MDCK cell expansion: Mardin-Darbin canine kidney cells were maintained in modified Eagle's medium (MEM) containing 10% of fetal bovine serum (FBS). When the confluent monolayer reached 80 to 90%, cells were washed twice with phosphate buffer solution (PBS) and treated with trypsin for 14 minutes. Then, MDCK cell were resuspended in 10 ml of MEM 10% media and counted. For new flask passages, the cells were diluted 1 in 3 and seeded in 15 ml of media. For inhibition assays, MDCK cells were seed at 2×10⁴ MDCK cells/well in a 96 well plate. In both cases, cells were incubated at 37° C. and 5% CO₂.

Note: MDCK cells should be in low passages and test negative for mycoplasma and other pathogens before use. For other respiratory viruses testing cells, time, and virus concentration varies.

Flu virus stock preparation: 5×10⁵ MDCK cells were seeded in T75 flask, after 24 hours of incubation at 37° C. and 5% CO₂, cells were washed twice and inoculated with Flu virus strain A/Puerto Rico/8/1934 (PR8) at 0.01 multiplicity of infection (MOI) for 1 hour at 37° C. and 5% CO₂. After incubation, 10 ml of MEM 0.35% of Bovine serum albumin (BSA), 1 μg/ml TPCK-trypsin was added. Supernatant were collected at 24, 48, and 72 hours post infection, aliquoted and kept at −80° C. for viral titration.

Viral titration by plaque forming units: To determine the Plaque formation units (PFU), 10-fold viral dilutions were prepared from the viral stocks. MDCK cells were seeded in 6 well plates. 24 hours later, when the monolayer reached between 80 and 90%, cells were washed twice with PBS and inoculated with viral stock dilutions. Incubated for one hour and cover with semisolid media containing 10 ml of MEM 0.35% of BSA, 1 μg/ml TPCK-trypsin, and 0.3% agarose. After 72 hours incubation at 37° C. and 5% CO₂, all plaques were stained with crystal violet as described below. The PFU were determined by counting plaques in the dilution producing around 50 visible plaques and then applying the following formula:

PFU/ml=(N×10^{X})/V where N is the number of plaques, X is the dilution counted, and V is the virus volume used per well.

Antiviral compounds: Favipiravir, Mycophenolate, and Viramidine were prepared as 10 mM stocks in dimethyl sulfoxide (DMSO) and were soluble when diluted in various reaction mixtures and cell culture media. Favipiravir was tested using concentrations ranging from 200 μM to 1.56 μM, with an estimated IC₅₀ of 25 μM when used alone. Mycophenolate was tested in concentrations between 2 μM and 0.25 μM with an estimated IC₅₀ of 1.422 μM when used alone. Viramidine tested concentration were between 200 and 1.56 μM, with an estimated IC₅₀ of 94.7 μM when used alone. Also, combination of Favipiravir with each of the other drugs were tested.

MDCK viral infection: After 24 hours, those cells in 96 well plates were wash twice with PBS and infected with PR8 Flu virus at 0.01 multiplicity of infection MOI for 1 hour at 37° C. and 5% CO₂. After incubation, the inoculum was carefully aspirated and replaced with MEM 0.35% of Bovine serum albumin (BSA), 1 μg/ml TPCK-trypsin, and different concentration of the antiviral drug.

Crystal violet staining: After 48 hours of incubation, the media was aspirated and MDCK cells washed once with PBS. The infectious virus was inactivated by filling each well with 4% paraformaldehyde and incubating 20 minutes at room temperature (RT). Then, the liquid was aspirated, and the wells were filled with crystal violet. After 10 minutes of incubation at RT, the crystal violet was carefully washed with tap water and the plates were dried overnight. At this point, the EC₅₀ could be visually estimated.

OD readings: Dried plate were treated with 1% SDS to solubilize the stain. This process took 20 minutes with constant shaking until the color was uniform. Then, the optical densities (OD) of each well were measured at 590 nm.

IC₅₀ calculation: To calculate the IC₅₀ concentrations, the log of the μM drug concentrations was compared with the % of inhibition based on the OD readings using excel software. IC₅₀ was calculated using a four-parameter logistic nonlinear regression model by GraphPad Prism.

Synergistic interaction analysis: The Synergy Finder2.0 was used to calculate the highest single agent (HSA) synergy score of two-drug combinations from different pairwise combinations. Areas with synergy score less than −10: the interaction between two drugs is likely to be antagonistic: from −10 to 10: the interaction between two drugs is likely to be additive: larger than 10: the interaction between two drugs is likely to be synergistic.

### INDUSTRIAL APPLICABILITY

The present combination product inhibits CoV2 replication through one or more mechanisms of action and increases potency of nucleoside and nucleotide analog drugs through inhibition of cellular enzymes involved in purine nucleotide biosynthesis and it is useful in treatment and prevention of SARS-CoV-2 virus infection.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A combination product comprising:
(i) Favipiravir and
(ii) IMPDH inhibitor
for the treatment of infection caused by an RNA virus.

2. The combination product of claim 1, wherein Favipiravir is a nucleoside base (Favipiravir), favipiravir ribonucleotide, or nucleotide triphosphate (Favipiravir-NTP) 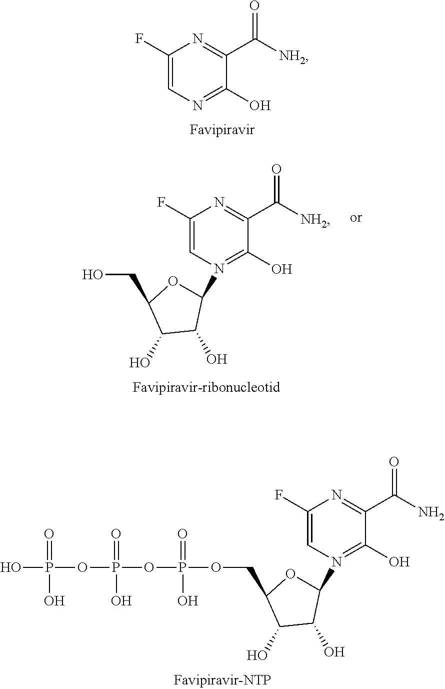
or a pharmaceutically acceptable salt, solvate, or tautomer thereof.

3. The combination product of claim 1, wherein the IMPDH inhibitor is selected from:
ribavirin;
mycophenolic acid (MPA);
merimepodib (VX-497);
viramidine;
mizoribine;
5-ethyl-1-beta-d-ribo-furanosylimidazole-4-carboamide (EICAR);
mycophenolate mofetil;
6-chloropurin riboside;
3-deazaguanosine;
2-vinylinosines;
thiazole-4-carboxamide dinucleotide;
tiazofurin;
2-(fluorovinyl) inosine monophosphate;
selenazofurin;
benzamidine riboside; and
VX-148;
or a pharmaceutically acceptable salt, stereoisomer, solvate, or tautomer thereof.

4. The combination product of claim 1, wherein the IMPDH inhibitor is mycophenolate mofetil or viramidine: 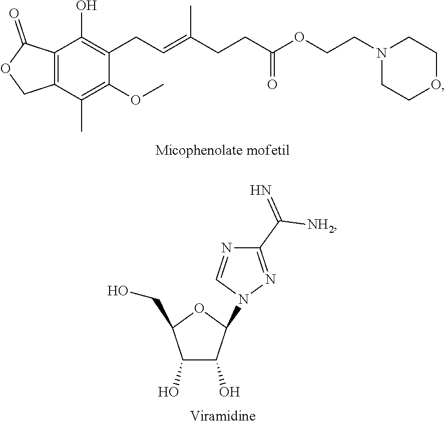 [SMILES: COc1c(C)c2c(c(O)c1C/C=C(\C)CCC(=O)OCCN1CCOCC1)C(=O)OC2 ; N=C(N)c1ncn([C@@H]2O[C@H](CO)[C@@H](O)[C@H]2O)n1]
or a pharmaceutically acceptable salt, stereoisomer, solvate, or tautomer thereof.

5. The combination product comprising:
(i) favipiravir and
(ii) mycophenolate mofetil.

6. The combination product comprising:
(i) favipiravir and
(ii) viramidine.

7. The combination product any one of preceding claims, wherein the combination product is a kit of parts comprising components (i) and (ii), for sequential, separate, or simultaneous use.

8. The combination product according to any one of claims 1-6, wherein the combination product is a pharmaceutical composition comprising components (i) and (ii) and a pharmaceutically acceptable carrier.

9. A pharmaceutical composition comprising (i) favipiravir or pharmaceutically acceptable salt, solvate, or tautomer thereof; (ii) IMPDH inhibitor or pharmaceutically acceptable salt, solvate, or tautomer thereof and a pharmaceutically acceptable carrier.

10. A pharmaceutical composition of claim 9 comprising (i) favipiravir or pharmaceutically acceptable salt, solvate, or tautomer thereof: (ii) mycophenolate mofetil or pharmaceutically acceptable salt, solvate, or tautomer thereof and a pharmaceutically acceptable carrier.

11. A pharmaceutical composition of claim 9 comprising (i) favipiravir or pharmaceutically acceptable salt, solvate, or tautomer thereof: (ii) viramidine or pharmaceutically acceptable salt, solvate, or tautomer thereof and a pharmaceutically acceptable carrier.

12. The pharmaceutical composition of any one of claim 9-11, further comprising an additional pharmaceutically active agent.

13. A method of inhibiting of RNA replication of virus, comprising of administering to a subject a combination product of claim 1.

14. A method of treating a viral infection caused by an RNA virus, comprising of administering to a subject a combination product of claim 1.

15. A method of treating RNA viral infection of claim 14 wherein virus selected from Measles, Mumps, Respiratory Syncytial Virus, SARS-CoV-2, Metapneumovirus, Poliovirus, Chikungunya virus, Hepatitis A virus, Hepatitis C virus, Hepatitis E virus, West Nile virus, Zika virus, Dengue virus, Lassa Fever virus, Junin South American hemorrhagic fever virus, Marburg virus, Ebola virus, Norovirus (human caliciviruses) infections, Rift Valley Fever virus, Nipah virus, Hendra virus, HIV-1, HTLV-1, HTLV-2, rotavirus, seasonal and pandemic coronaviruses, Picobirnaviruses, Toroviruses, and Astroviruses comprising of administering to a subject in need of a treatment a combination product of any one of claims 1-8 or a pharmaceutical composition of any one of claims 9-12.

16. The method of claim 14, wherein viral infection is SARS-CoV-2 virus infection.

17. The method of any one of claims 13-16, wherein the subject is a mammal.

18. The method of claim 17, wherein the subject is a human.
